(19) United States
(12) Patent Application Publication
(10) Pub. No.: US 2004/0049354 A1
(43) Pub. Date: Mar. 11, 2004

(54) Method, system and computer software providing a genomic web portal for functional analysis of alternative splice variants

(75) Inventors: Ann Loraine, El Cerrito, CA (US); Melissa Cline, Santa Cruz, CA (US); Gregg A. Helt, Healdsburg, CA (US); Michael A. Siani-Rose, San Francisco, CA (US); David C. Kulp, Albany, CA (US); Ron T. Shigeta, Berkeley, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignees: Affymetrix, Inc.; a Corporation Organized under the laws of Delaware

(21) Appl. No.: 10/423,403

(22) Filed: Apr. 25, 2003

Related U.S. Application Data
(63) Continuation-in-part of application No. 10/065,856, filed on Nov. 26, 2002.
(63) Continuation-in-part of application No. 10/065,868, filed on Nov. 26, 2002.
(63) Continuation-in-part of application No. 10/328,818, filed on Dec. 23, 2002.
(63) Continuation-in-part of application No. 10/328,872, filed on Dec. 23, 2002.
(60) Provisional application No. 60/376,003, filed on Apr. 26, 2002.
(60) Provisional application No. 60/394,574, filed on Jul. 9, 2002.
(60) Provisional application No. 60/403,381, filed on Aug. 14, 2002.

(51) Int. Cl. G06F 19/00; G01N 33/48; G01N 33/50

(52) U.S. Cl. 702/19; 702/20

(57) ABSTRACT

A system for analyzing alternative splice variant sequences is described, comprising an input manager for receiving alternative splice variant sequences that are identified by one or more probe sets, a correlator that correlates functional domains with each of the alternative splice variant sequences and an associater that associates putative functions with the alternative splice variant sequences based upon a combination of the functional domains. A method for analyzing alternative splice variant sequences is also described, comprising the acts of receiving alternative splice variant sequences that are identified by one or more probe sets, correlating functional domains with the alternative splice variant sequences and associating putative functions with the alternative splice variant sequences based upon a combination of the functional domains.

### BACKGROUND

1. Field of the Invention

The present invention relates to the field of bioinformatics. In particular, the present invention relates to computer systems, methods, and products for providing genomic information over networks such as the Internet.

2. Related Art

Research in molecular biology, biochemistry, and many related health fields increasingly requires organization and analysis of complex data generated by new experimental techniques. These tasks are addressed by the rapidly evolving field of bioinformatics. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Baxevanis, eds., Wiley & Sons, Inc.; 2d ed., 2001), both of which are hereby incorporated herein by reference in their entireties. Broadly, one area of bioinformatics applies computational techniques to large genomic databases, often distributed over and accessed through networks such as the Internet, for the purpose of illuminating relationships among alternative splice variants, protein function, and metabolic processes.

### SUMMARY OF THE INVENTION

The expanding use of microarray technology is one of the forces driving the development of bioinformatics. In particular, microarrays and associated instrumentation and computer systems have been developed for rapid and large-scale collection of data about the expression of genes or expressed sequence tags (ESTs) in tissue samples. Data from experiments with genotyping microarrays may be used, among other things, to study genetic characteristics and to detect mutations relevant to genetic and other diseases or conditions. More specifically, the data gained through microarray experiments is valuable to researchers because, among other reasons, many disease states can potentially be characterized by differences in the expression levels of various genes, either through changes in the copy number of the genetic DNA or through changes in levels of transcription (e.g., through control of initiation, provision of RNA precursors, or RNA processing) of particular genes. Thus, for example, researchers use microarrays to answer questions such as: Which genes are expressed in cells of a malignant tumor but not expressed in either healthy tissue or tissue treated according to a particular regime? Which genes or ESTs are expressed in particular organs but not in others? Which genes or ESTs are expressed in particular species but not in others? How does the environment, drugs, or other factors influence gene expression? Data collection is only an initial step, however, in answering these and other questions. Researchers are increasingly challenged to extract biologically meaningful information from the vast amounts of data generated by microarray technologies, and to design follow-on experiments. A need exists to provide researchers with improved tools and information to perform these tasks.

Systems, methods, and computer program products are described herein to address these and other needs. A system for analyzing alternative splice variant sequences is described, comprising an input manager constructed and arranged to receive at least two alternative splice variant sequences, wherein the at least two alternative splice variant sequences are identified by one or more probe sets, a correlator constructed and arranged to correlate one or more functional domains with each of the at least two alternative splice variant sequences and an associater constructed and to associate one or more putative functions with each of the at least two alternative splice variant sequences based, at least in part, upon a combination of the one or more functional domains.

In accordance with another embodiment a system is described, comprising an input manager constructed and arranged to receive a plurality of probe set identifiers and associated intensity values, a determiner constructed and arranged to determine at least two alternative splice variant sequences based, at least in part, upon the one or more probe set identifiers and associated intensity values, a correlator constructed and arranged to correlate one or more functional domains with each of the at least two alternative splice variant sequences, an associater constructed and arranged to associate one or more putative functions with each of the at least two alternative splice variant sequences based, at least in part, upon a combination of the one or more functional domains and an output manager constructed and arranged to display the putative functions in one or more graphical user interfaces.

In accordance with another embodiment a system is described, comprising an input manager constructed and arranged to receive at least two alternative splice variant sequences, a correlator constructed and arranged to correlate one or more functional domains with each of the at least two alternative splice variant sequences, a analyzer constructed and arranged to compare one or more differences between each of the at least two alternative splice variant sequences based, at least in part, upon the one or more functional domains and an output manager constructed and arranged to display the one or more differences of each of the at least two alternative splice variant sequences in one or more graphical user interfaces.

In accordance with another embodiment a system is described, comprising an application server comprising an input manager constructed and arranged to receive at least two alternative splice variant sequences, wherein the at least two alternative splice variant sequences are identified by one or more probe sets, a correlator constructed and arranged to correlate one or more functional domains with each of the at least two alternative splice variant sequences and an associater constructed and to associate one or more putative functions with each of the at least two alternative splice variant sequences based, at least in part, upon a combination of the one or more functional domains and the system also comprises an internet server comprising an output manager constructed and arranged to display the putative functions in one or more graphical user interfaces.

In accordance with another embodiment a system is described, comprising means for receiving at least two alternative splice variant sequences, wherein the at least two alternative splice variant sequences are identified by one or more probe sets, means for correlating one or more functional domains with each of the at least two alternative splice variant sequences and means for associating one or more putative functions with each of the at least two alternative splice variant sequences based, at least in part, upon a combination of the one or more functional domains.

Furthermore, in accordance with some embodiments a method for analysis of alternative splice variant sequences is described, comprising the acts of receiving at least two alternative splice variant sequences, wherein the at least two alternative splice variant sequences are identified by one or more probe sets, correlating one or more functional domains with each of the at least two alternative splice variant sequences and associating one or more putative functions with each of the at least two alternative splice variant sequences based, at least in part, upon a combination of the one or more functional domains.

In accordance with another embodiment, a method is described, comprising the acts of receiving a plurality of probe set identifiers and associated intensity values, determining at least two alternative splice variant sequences based, at least in part, upon the one or more probe set identifiers and associated intensity values, correlating one or more functional domains with each of the at least two alternative splice variant sequences, associating one or more putative functions with each of the at least two alternative splice variant sequences based, at least in part, upon a combination of the one or more functional domains and displaying the putative function in one or more graphical user interfaces.

In accordance with another embodiment, a method is described, comprising the acts of receiving at least two alternative splice variant sequences, correlating one or more functional domains with each of the at least two alternative splice variant sequences, comparing one or more differences between each of the at least two alternative splice variant sequences based, at least in part, upon the one or more functional domains, and displaying the one or more differences of each of the at least two alternative splice variant sequences in one or more graphical user interfaces.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost one or two digits of a reference numeral indicate the number of the figure in which the referenced element first appears (for example, the element 180 appears first in FIG. 1; element 1120 appears first in FIG. 11). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a probe-array analysis system including an illustrative scanner and an illustrative computer system;

FIG. 2 is a functional block diagram of one embodiment of probe-array analysis applications as illustratively stored for execution in system memory of the computer system of FIG. 1;

FIG. 3 is a functional block diagram of a conventional system for obtaining genomic information over the Internet;

FIG. 4 is a functional block diagram of one embodiment of a genomic portal coupled over the Internet to remote databases and web pages and to clients including networks having user computer systems including that of FIG. 1;

FIG. 5 is a functional block diagram of one embodiment of the genomic portal of FIG. 4 including illustrative embodiments of a database server, portal application computer system, and portal-side Internet server;

FIG. 6 is a simplified graphical representation of one embodiment of computer application platforms for implementing the genomic portal of FIGS. 4 and 5 in communication with clients such as those shown in FIG. 4;

FIG. 7 is a flow chart of one embodiment of a method for providing a user with web pages displaying data related to functional analysis of alternative splice variants and/or experiment data;

FIG. 8 is a functional block diagram of one embodiment of a user-service manager application as may be executed on the portal application computer system of FIG. 5;

FIG. 9 is a simplified graphical representation of one embodiment of a local genomic database such as may be accessed by the database server of FIG. 5;

FIG. 10 is a functional block diagram of one embodiment of a correlator such as may be included in the user-service manager application of FIG. 8;

FIG. 11 is a functional block diagram of one embodiment of a alternative splice variants analyzer as may be included in the user-service manager application of FIG. 8; and

FIG. 12 is a graphical representation of one embodiment of a graphical user interface suitable for providing data related to functional analysis of alternative splice variants, alternative transcript variants and/or experiment data generated by alternative splice variants analyzer of FIG. 11.

### DETAILED DESCRIPTION

The present invention has many preferred embodiments that, in some instances, may include material incorporated from patents, applications and other references for details known to those of the art. When a patent or patent application is referred to below, it should be understood that it is incorporated by reference in its entirety for all purposes. As used in this application, the singular form “a,” “an,” and “the” include plural references unless the context clearly dictates otherwise. For example, the term “an agent” includes a plurality of agents, including mixtures thereof. An individual is not limited to a human being but may also be other organisms including but not limited to mammals, plants, bacteria, or cells derived from any of the above.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This principle applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques may be had by reference to the examples herein. However, other equivalent conventional procedures may, of course, also be used. Such conventional techniques and descriptions may be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, In Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The practice of the present invention may also employ conventional biology methods, software, and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, and other known devices or media and those that may be developed in the future. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2^{nd} ed., 2001).

As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both of which are hereby incorporated by reference for all purposes.

Computer software products may be written in any of various suitable programming languages, such as C, C++, Fortran and Java (Sun Microsystems). The computer software product may be an independent application with data input and data display modules. Alternatively, the computer software products may be classes that may be instantiated as distributed objects. The computer software products may also be component software such as Java Beans (Sun Microsystems), Enterprise Java Beans (EJB), Microsoft® COM/DCOM, etc.

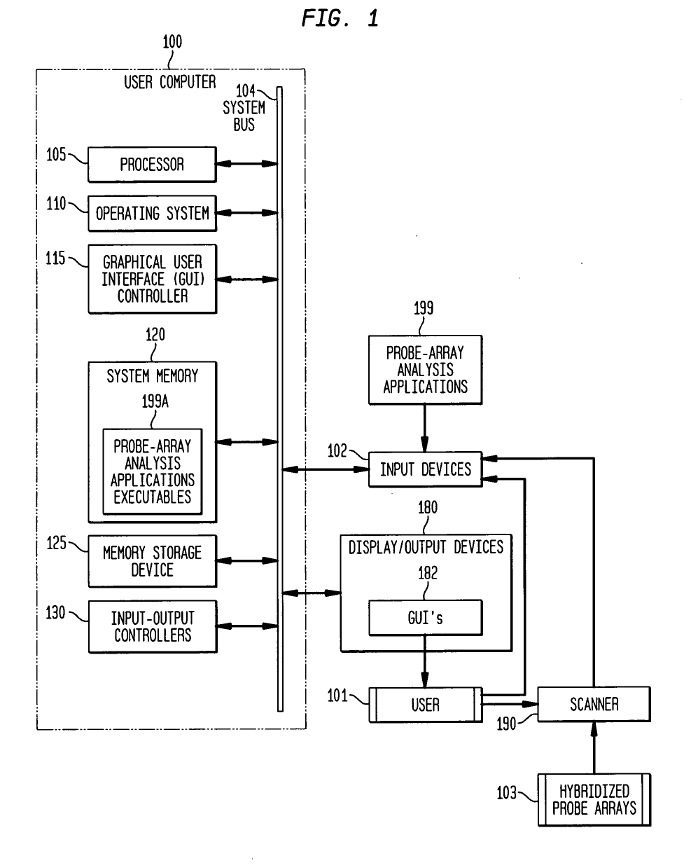

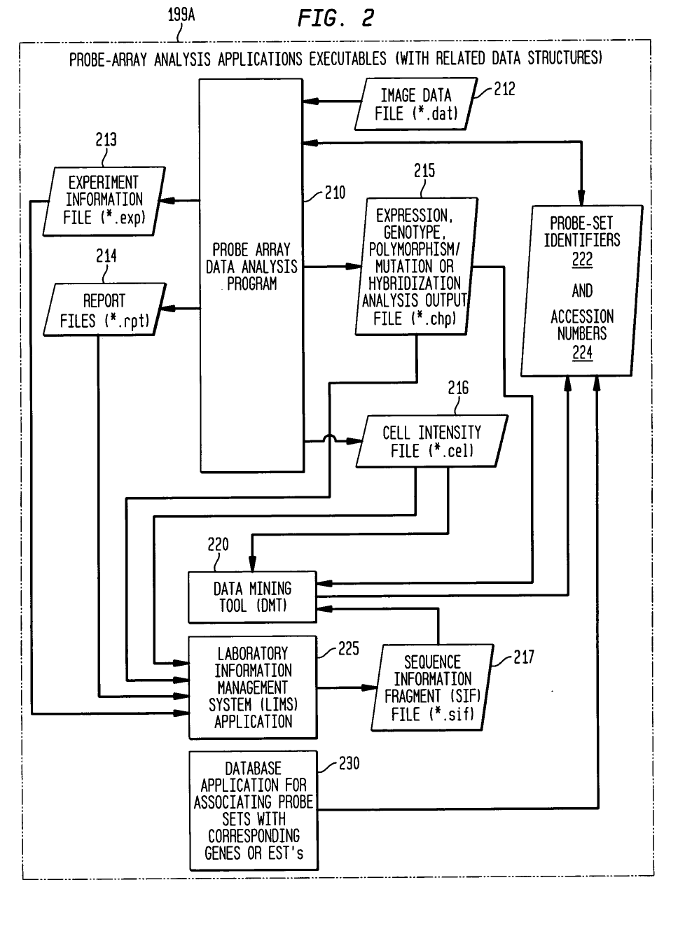

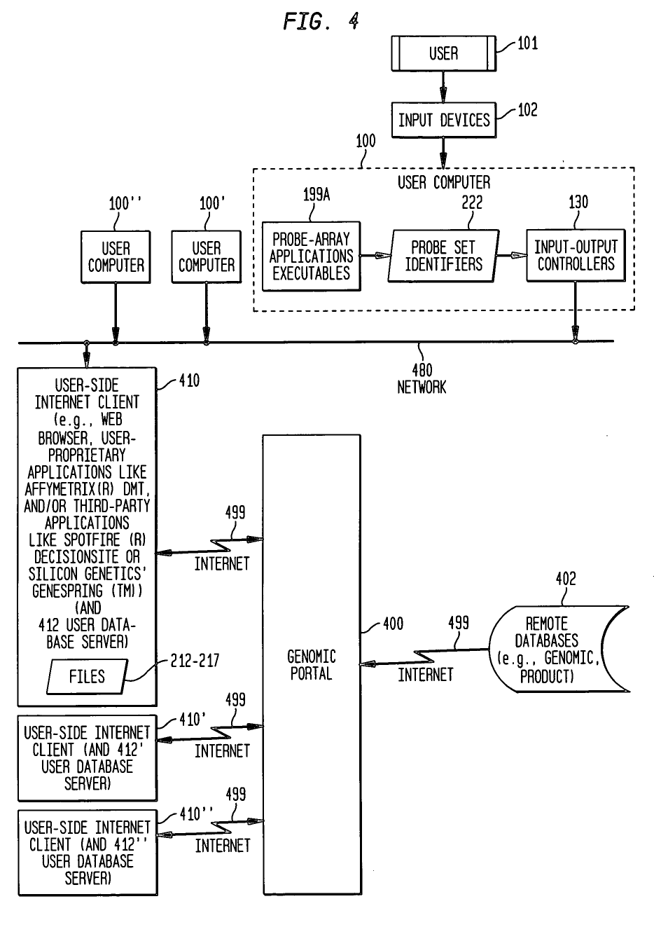

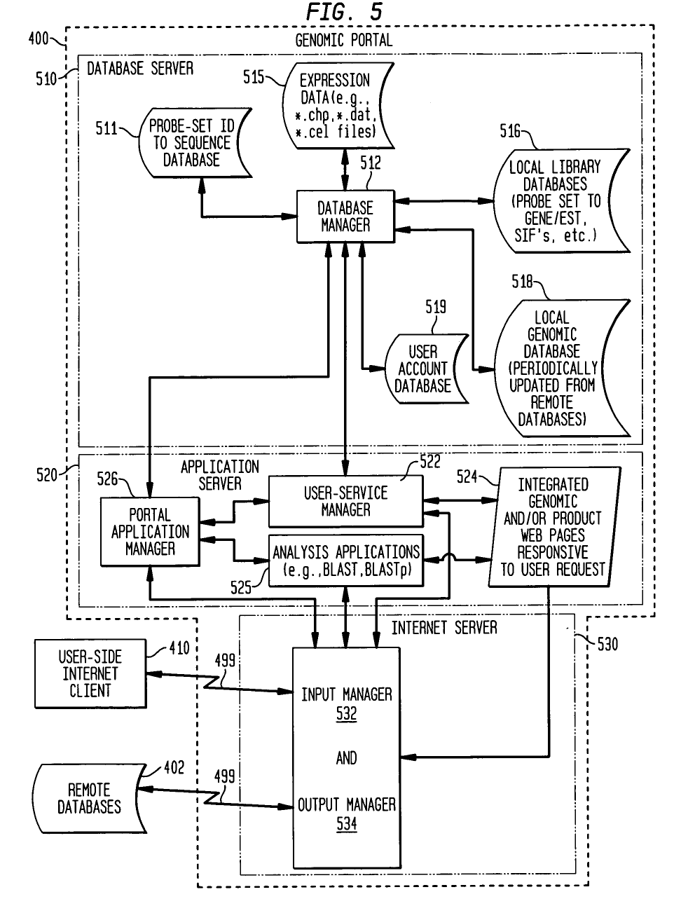

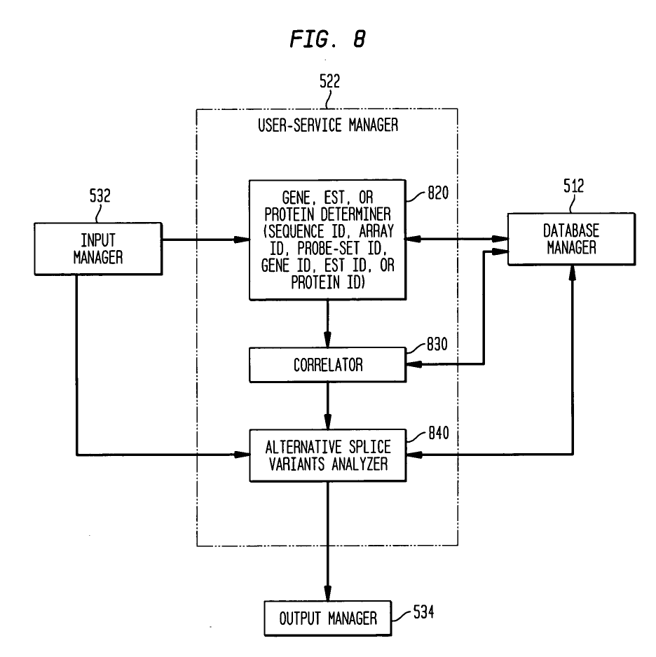

Systems, methods, and computer products are now described with reference to an illustrative embodiment referred to as genomic portal 400. Portal 400 is shown in an Internet environment in FIG. 4, and is illustrated in greater detail in FIGS. 5 through 19. In a typical implementation, portal 400 may be used to provide a user with information related to results from experiments with probe arrays. The experiments often involve the use of scanning equipment to detect hybridization of probe-target pairs, and the analysis of detected hybridization by various software applications, as now described in relation to FIGS. 1 and 2.

Probe Arrays 103: Various techniques and technologies may be used for synthesizing dense arrays of biological materials on or in a substrate or support to form microarrays, including spotted arrays. For example, Affymetrix® GeneChip® arrays are synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. Pat. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285, which are all incorporated herein by reference in their entireties for all purposes.

Patents that describe synthesis techniques in specific embodiments include U.S. Pat. Nos. 6,486,287, 6,147,205, 6,262,216, 6,310,189, 5,889,165, 5,959,098, and 5,412,087, all hereby incorporated by reference in their entireties for all purposes. Nucleic acid arrays are described in many of the above patents, but the same techniques generally may be applied to polypeptide arrays or arrays of other biochemical molecules.

Generally speaking, an “array” typically includes a collection of molecules that can be prepared either synthetically or biosynthetically. The molecules in the array may be identical, they may be duplicative, and/or they may be different from each other. The array may assume a variety of formats, e.g., libraries of soluble molecules; libraries of compounds tethered to resin beads, silica chips, or other solid supports; and other formats.

The terms “solid support,” “support,” and “substrate” may in some contexts be used interchangeably and may refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches or wells, or other separation members or elements. In some embodiments, the solid support(s) may take the form of beads, resins, gels, microspheres, or other materials and/or geometric configurations.

Generally speaking, a “probe” typically is a molecule that can be recognized by a particular target. To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what is referred to herein as the “target.”

A target is a molecule that has an affinity for a given probe. Targets may be naturally-occurring or man-made molecules. Also, they can be employed in their unaltered state or as aggregates with other species. The samples or targets are processed so that, typically, they are spatially associated with certain probes in the probe array. For example, one or more tagged targets may be distributed over the probe array.

Targets may be attached, covalently or noncovalently, to a binding member, either directly or via a specific binding substance. Examples of targets that can be employed in accordance with this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. Typically, a “probe-target pair” is formed when two macromolecules have combined through molecular recognition to form a complex.

The probes of the arrays in some implementations comprise nucleic acids that are synthesized by methods including the steps of activating regions of a substrate and then contacting the substrate with a selected monomer solution. The term “monomer” generally refers to any member of a set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone. In addition, the terms “biopolymer” and “biological polymer” generally refer to repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer. Related to the term “biopolymer” is the term “biomonomer” that generally refers to a single unit of biopolymer, or a single unit that is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

As used herein, nucleic acids may include any polymer or oligomer of nucleosides or nucleotides (polynucleotides or oligonucleotides) that include pyrimidine and/or purine bases, preferably cytosine, thymine, and uracil, and adenine and guanine, respectively. An “oligonucleotide” or “polynucleotide” is a nucleic acid ranging from at least 2, preferably at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide in accordance with the present invention may be peptide nucleic acid (PNA) in which the constituent bases are joined by peptides bonds rather than phosphodiester linkage, as described in Nielsen et al., Science 254:1497-1500 (1991); Nielsen, Curr. Opin. Biotechnol., 10:71-75 (1999), both of which are hereby incorporated by reference herein. The invention also encompasses situations in which there is a nontraditional base pairing such as Hoogsteen base pairing that has been identified in certain tRNA molecules and postulated to exist in a triple helix. “Polynucleotide” and “oligonucleotide” may be used interchangeably in this application.

Additionally, nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

As noted, a nucleic acid library or array typically is an intentionally created collection of nucleic acids that can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids that can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. Nucleic acid arrays that are useful in the present invention include those that are commercially available from Affymetrix, Inc. of Santa Clara, Calif., under the registered trademark “GeneChip®.” Example arrays are shown on the website at affymetrix.com.

In some embodiments, a probe may be surface immobilized. Examples of probes that can be investigated in accordance with this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. As non-limiting examples, a probe may refer to a nucleic acid, such as an oligonucleotide, capable of binding to a target nucleic acid of complementary sequence through one or more types of chemical bonds, usually through complementary base pairing, usually through hydrogen bond formation. A probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, or any ligands for detecting its binding partners. Probes of other biological materials, such as peptides or polysaccharides as non-limiting examples, may also be formed. For more details regarding possible implementations, see U.S. Pat. No. 6,156,501, hereby incorporated by reference herein in its entirety for all purposes. When referring to targets or probes as nucleic acids, it should be understood that these are illustrative embodiments that are not to limit the invention in any way.

Furthermore, to avoid confusion, the term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described herein may also be employed with respect to other parallel assay devices. For example, these functions and methods may be applied with respect to probe-set identifiers that identify probes immobilized on or in beads, optical fibers, or other substrates or media.

In accordance with some implementations, some targets hybridize with probes and remain at the probe locations, while non-hybridized targets are washed away. These hybridized targets, with their tags or labels, are thus spatially associated with the probes. The term “hybridization” refers to the process in which two single-stranded polynucleotides bind non-covalently to form a stable double-stranded polynucleotide. The term “hybridization” may also refer to triple-stranded hybridization, which is theoretically possible. The resulting (usually) double-stranded polynucleotide is a “hybrid.” The proportion of the population of polynucleotides that forms stable hybrids is referred to herein as the “degree of hybridization.” Hybridization probes usually are nucleic acids (such as oligonucleotides) capable of binding in a base-specific manner to a complementary strand of nucleic acid. Such probes include peptide nucleic acids, as described in Nielsen et al., Science 254:1497-1500 (1991) or Nielsen Curr. Opin. Biotechnol., 10:71-75 (1999) (both of which are hereby incorporated herein by reference), and other nucleic acid analogs and nucleic acid mimetics. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entireties for all purposes.

The present invention also contemplates signal detection of hybridization between probes and targets in certain preferred embodiments. See U.S. Pat. Nos. 5,143,854, 5,578,832; 5,631,734; 5,936,324; 5,981,956; 6,025,601 incorporated above and in U.S. Pat. Nos. 5,834,758, 6,141,096; 6,185,030; 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

A system and method for efficiently synthesizing probe arrays using masks is described in U.S. patent application Ser. No. 09/824,931, filed Apr. 3, 2001, that is hereby incorporated by reference herein in its entirety for all purposes. A system and method for a rapid and flexible microarray manufacturing and online ordering system is described in U.S. Provisional Patent Application Serial No. 60/265,103 filed Jan. 29, 2001, that also is hereby incorporated herein by reference in its entirety for all purposes. Systems and methods for optical photolithography without masks are described in U.S. Pat. No. 6,271,957 and in U.S. patent application Ser. No. 09/683,374 filed Dec. 19, 2001, both of which are hereby incorporated by reference herein in their entireties for all purposes.

As noted, various techniques exist for depositing probes on a substrate or support. For example, “spotted arrays” are commercially fabricated, typically on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer and 427™ Arrayer are devices that deposit densely packed arrays of biological materials on microscope slides in accordance with these techniques. Aspects of these and other spot arrayers are described in U.S. Pat. Nos. 6,040,193 and 6,136,269 and in PCT Application No. PCT/US99/00730 (International Publication Number WO 99/36760) incorporated above and in U.S. patent application Ser. No. 09/683,298 hereby incorporated by reference in its entirety for all purposes. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Other implementations of the jetting technique may use devices such as syringes or piezo electric pumps to propel the biological material. It will be understood that the foregoing are non-limiting examples of techniques for synthesizing, depositing, or positioning biological material onto or within a substrate. For example, although a planar array surface is preferred in some implementations of the foregoing, a probe array may be fabricated on a surface of virtually any shape or even a multiplicity of surfaces. Arrays may comprise probes synthesized or deposited on beads, fibers such as fiber optics, glass, silicon, silica or any other appropriate substrate, see U.S. Pat. No. 5,800,992 referred to and incorporated above and U.S. Pat. Nos. 5,770,358, 5,789,162, 5,708,153 and 6,361,947 all of which are hereby incorporated in their entireties for all purposes. Arrays may be packaged in such a manner as to allow for diagnostics or other manipulation in an all inclusive device, see for example, U.S. Pat. Nos. 5,856,174 and 5,922,591 hereby incorporated in their entireties by reference for all purposes.

Probes typically are able to detect the expression of corresponding genes or ESTs by detecting the presence or abundance of mRNA transcripts present in the target. This detection may, in turn, be accomplished in some implementations by detecting labeled cRNA that is derived from cDNA derived from the mRNA in the target.

The terms “mRNA” and “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Thus, MRNA derived samples include, but are not limited to, MRNA transcripts of the gene or genes, CDNA reverse transcribed from the mRNA, cRNA transcribed from the cDNA, DNA amplified from the genes, RNA transcribed from amplified DNA, and the like.

In general, a group of probes, sometimes referred to as a probe set, contains subsequences in unique regions of the transcripts and does not correspond to a full gene sequence. Further details regarding the design and use of probes and probe sets are provided in PCT Application Serial No. PCT/US 01/02316, filed Jan. 24, 2001 incorporated above; and in U.S. Pat. No. 6,188,783 and in U.S. patent application Ser. No. 09/721,042, filed on Nov. 21, 2000, Ser. No. 09/718,295, filed on Nov. 21, 2000, Ser. No. 09/745,965, filed on Dec. 21, 2000, and Ser. No. 09/764,324, filed on Jan. 16, 2001, all of which patent and patent applications are hereby incorporated herein by reference in their entireties for all purposes.

Scanner 190: FIG. 1 is a functional block diagram of a system that is suitable for, among other things, analyzing probe arrays that have been hybridized with labeled targets. Representative hybridized probe arrays 103 of FIG. 1 may include probe arrays of any type, as noted above. Labeled targets in hybridized probe arrays 103 may be detected using various commercial devices, referred to for convenience hereafter as “scanners.” An illustrative device is shown in FIG. 1 as scanner 190. In some implementations, scanners image the targets by detecting fluorescent or other emissions from the labels, or by detecting transmitted, reflected, or scattered radiation. These processes are generally and collectively referred to hereafter for convenience simply as involving the detection of “emissions.” Various detection schemes are employed depending on the type of emissions and other factors. A typical scheme employs optical and other elements to provide excitation light and to selectively collect the emissions. Also included in some implementations are various light-detector systems employing photodiodes, charge-coupled devices, photomultiplier tubes, or similar devices to register the collected emissions.

Methods and apparatus for signal detection and processing of intensity data are disclosed in, for example, U.S. Pat. Nos. 5,143,854, 5,578,832, 5,631,734, 5,800,992, 5,834,758, 5,856,092, 5,936,324, 5,981,956, 6,025,601, 6,090,555, 6,141,096, 6,185,030, 6,201,639, 6,207,960, 6,218,803, 6,225,625, in PCT Application PCT/JS99/06097 (published as WO99/47964) incorporated above, and in U.S. Pat. Nos. 5,547,839, 5,902,723, 6,171,793, 6,207,960, 6,252,236, 6,335,824, 6,490,533, 6,472,671, 6,403,320, and 6,407,858 each of which is hereby incorporated by reference in its entirety for all purposes. Other scanners or scanning systems are described in U.S. patent application Ser. No. 09/682,837 filed Oct. 23, 2001; Ser. No. 09/683,216 filed Dec. 3, 2001; Ser. No. 09/683,217 filed Dec. 3, 2001; Ser. No. 09/683,219 filed Dec. 3, 2001; and Ser. No. 10/389,194, filed Mar. 14, 2003, each of which is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,974,164, 6,090,555, 6,188,783 incorporated above and U.S. Pat. Nos. 5,733,729, 6,066,454, 6,185,561, 6,223,127, 6,229,911 and 6,308,170, hereby incorporated herein in their entireties for all purposes.

Scanner 190 provides data representing the intensities (and possibly other characteristics, such as color) of the detected emissions, as well as the locations on the substrate where the emissions were detected. The data typically are stored in a memory device, such as system memory 120 of user computer 100, in the form of a data file or other data storage form or format. One type of data file, such as image data file 212 shown in FIG. 2, typically includes intensity and location information corresponding to elemental sub-areas of the scanned substrate. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known. Two examples of image data are data files in the form *.dat or *.tif as generated respectively by Affymetrix® Microarray Suite or Affymetrix® GeneChip® Operating Software based on images scanned from GeneChip® arrays, and by Affymetrix® Jaguar™ software based on images scanned from spotted arrays.

Probe-Array Analysis Applications 199: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729 and in U.S. Pat. No. 5,837,832, noted and incorporated above.

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390, and PCT/US 01/2 26297, and in U.S. patent application Ser. Nos. 09/681,819, 09/682,071, 09/682,074, and 09/682,076, the Microarray Suite application from Affymetrix, Inc., aspects of which are described in U.S. patent application Ser. Nos. 09/683,912, 10/219,503, 10/219,882, and 10/370,442, and the GeneChip® Operating Software from Affymetrix, Inc., aspects of which are described in U.S. Provisional Patent Application No. 60/442,684, all of which are hereby incorporated herein by reference in their entireties for all purposes. For example, image data in image data file 212 may be operated upon to generate intermediate results such as so-called cell intensity files (*.cel) and chip files (*.chp), generated by Microarray Suite or GeneChip® Operating Software or spot files (*.spt) generated by Jaguar™ software. For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly. In the illustrative case in which image data file 212 is derived from a GeneChip® probe array, and in which Microarray Suite or GeneChip® Operating Software generates cell intensity file 216, file 216 may contain, for each probe scanned by scanner 190, a single value representative of the intensities of pixels measured by scanner 185 for that probe. Thus, this value is a measure of the abundance of tagged cRNA's present in the target that hybridized to the corresponding probe. Many such cRNA's may be present in each probe, as a probe on a GeneChip® probe array may include, for example, millions of oligonucleotides designed to detect the cRNA's. The resulting data stored in the chip file may include degrees of hybridization, absolute and/or differential (over two or more experiments) expression, genotype comparisons, detection of polymorphisms and mutations, and other analytical results. In another example, in which executables 199A includes image data from a spotted probe array, the resulting spot file includes the intensities of labeled targets that hybridized to probes in the array. Further details regarding cell files, chip files, and spot files are provided in U.S. patent application Ser. Nos. 09/683,912, 10/219,503, 10/219,882, and 10/370,442, incorporated by reference above.

In the present example, in which executables 199A may include aspects of Affymetrix® Microarray Suite or GeneChip® Operating Software, the chip file is derived from analysis of the cell file combined in some cases with information derived from library files (not shown) that specify details regarding the sequences and locations of probes and controls. Laboratory or experimental data may also be provided to the software for inclusion in the chip file. For example, an experimenter and/or automated data input devices or programs (not shown) may provide data related to the design or conduct of experiments. As a non-limiting example related to the processing of an Affymetrix® GeneChip® probe array, the experimenter may specify an Affymetrix catalog or custom chip type (e.g., Human Genome U95Av2 chip) either by selecting from a predetermined list presented by Microarray Suite or GeneChip® Operating Software or by scanning a bar code related to a chip to read its type. Microarray Suite or GeneChip® Operating Software may associate the chip type with various scanning parameters stored in data tables including the area of the chip that is to be scanned, the location of chrome borders on the chip used for auto-focusing, the wavelength or intensity of laser light to be used in reading the chip, and so on. Other experimental or laboratory data may include, for example, the name of the experimenter, the dates on which various experiments were conducted, the equipment used, the types of fluorescent dyes used as labels, protocols followed, and numerous other attributes of experiments. As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer, such as user database server 412 of FIG. 4, configured to manage information from large numbers of experiments. Data analysis program 210 may also generate various types of plots, graphs, tables, and other tabular and/or graphical representations of analytical data such as contained in file 215. As will be appreciated by those skilled in the relevant art, the preceding and following descriptions of files generated by executables 199A are exemplary only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool, illustrated in FIG. 2 as Data Mining Tool 220 and described in U.S. patent application Ser. No. 09/683,980 which is hereby incorporated herein by reference in its entirety for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS), aspects of which illustrated as Laboratory Information Management System Application 225 and are described in U.S. patent application Ser. No. 09/682,098 hereby incorporated by reference herein in its entirety for all purposes. In addition, various proprietary databases accessed by database management software, such as the Affymetrix® EASI (Expression Analysis Sequence Information) database and database software, provide researchers with associations between probe sets and gene or EST identifiers.

For convenience of reference, these types of computer software applications (i.e., for acquiring and processing image files, data mining, data management, and various database and other applications related to probe-array analysis) are generally and collectively represented in FIG. 1 as probe-array analysis applications 199. FIG. 2 is a functional block diagram of probe-array analysis applications 199 as illustratively stored for execution (as executable code 199A corresponding to applications 199) in system memory 120 of user computer 100 of FIG. 1.

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases, such as Affymetrix® LIMS or Affymetrix® Data Mining Tool (DMT), to be executed from a database server such as user database server 412 of FIG. 4. Alternatively, LIMS, DMT, and/or other applications may be executed from computer 100, but some or all of the databases upon which those applications operate may be stored for common access on server 412 (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network is represented in FIG. 4 by the connection of user computer 100 to user database server 412 (and to user-side Internet client 410, which may be the same computer) via network cable 480. Similarly, scanner 190 (or multiple scanners) may be made available to a network of users over cable 480 both for purposes of controlling scanner 190 and for receiving data input from it.

In some implementations, it may be convenient for user 101 to group probe-set identifiers 222 for batch transfer of information or to otherwise analyze or process groups of probe sets together. For example, as described below, user 101 may wish to obtain annotation information via portal 400 related to one or more probe sets identified by their respective probe-set identifiers. Rather than obtaining this information serially, user 101 may group probe sets together for batch processing. Various known techniques may be employed for associating probe-set identifiers, or data related to those identifiers, together. For instance, user 101 may generate a tab delimited *.txt file including a list of probe-set identifiers for batch processing. This file or another file or data structure for providing a batch of data (hereafter referred to for convenience simply as a “batch file”), may be any kind of list, text, data structure, or other collection of data in any format. The batch file may also specify what kind of information user 101 wishes to obtain with respect to all, or any combination of, the identified probe sets. In some implementations, user 101 may specify a name or other user-specified identifier to represent the group of probe-set identifiers specified in the text file or otherwise specified by user 101. This user-specified identifier may be stored by one of executables 199A, or by elements of portal 400 described below, so that user 101 may employ it in future operations rather than providing the associated probe-set identifiers in a text file or other format. Thus, for example, user 101 may formulate one or more queries associated with a particular user-specified identifier, resulting in a batch transfer of information from portal 400 to user 101 related to the probe-set identifiers that user 101 has associated with the user-specified identifier. Alternatively, user 101 may initiate a batch transfer by providing the text file of probe-set identifiers. In any of these cases, user 101 may formulate queries to obtain, in a single batch operation, probe set records, lists of probe sets sorted into functional groups, protein functional domain information, sequence homology information, metabolic pathway information, BLAST similarity searches, array content information, and any other information available via portal 400. Similarly, user 101 may provide information, such as laboratory or experimental information, related to a number of probe sets by a batch operation rather than serial ones. The probe sets may be grouped by experiments, by similarity of probe sets (e.g., probe sets representing genes having similar annotations, such as related to transcription regulation), or any other type of grouping. For example, user 101 may assign a user-specified identifier (e.g., “experiments of January 1”) to a series of experiments and submit probe-set identifiers in user-selected categories (e.g., identifying probe sets that were up-regulated by a specified amount) and provide the experimental information to portal 400 for data storage and/or analysis.

User Computer 100: User computer 100, shown in FIG. 1, may be a computing device specially designed and configured to support and execute some or all of the functions of probe array applications 199. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 101, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

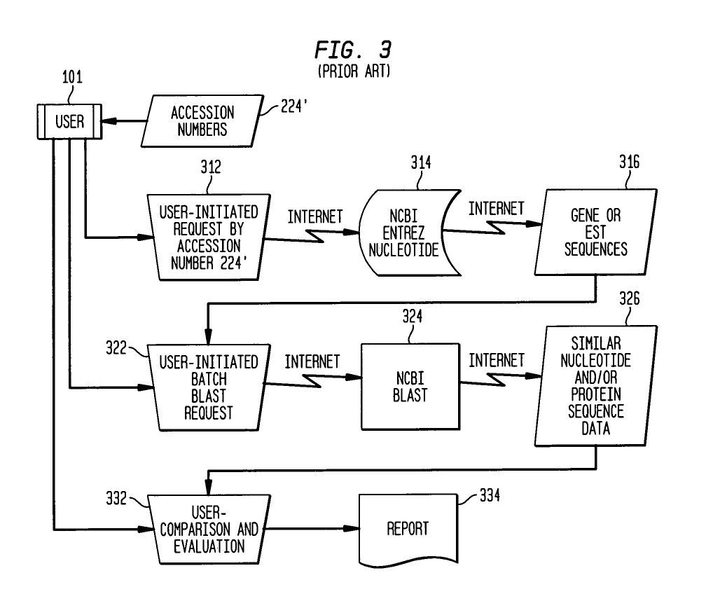

Conventional Techniques for Obtaining Genomic Data: A number of conventional approaches for obtaining genomic data over the Internet are available, some of which are described in the book edited by Ouelette and Baxevanis, incorporated by reference above. FIG. 3 is a functional block diagram representing one simplified example. As shown in FIG. 3, user 101 may consult any of a number of public or other sources to obtain accession numbers 224′. As represented by manual operation 312, user 101 initiates request 312 by accessing through any web browser the Internet web site of the National Center for Biotechnology Information (NCBI) of the National Library of Medicine and the National Institutes of Health (as of November 2002, accessible at the Internet URL http://www.ncbi.nlm.nih.gov/). In particular, user 101 may access the Entrez search and retrieval system that provides information from various databases at NCBI. These databases provide information regarding nucleotide sequences, protein sequences, macromolecular structures, whole genomes, and publication data related thereto. It is illustratively assumed that user 101 accesses in this manner NCBI Entrez nucleotide database 314 and receives information including gene or EST sequences 316. Particularly if accession numbers 224′ represents a large number (e.g., one hundred) of ESTs or genes of interest, as may easily be the case following analysis of probe array experiments, the tasks thus far described may take significant time, perhaps hours.

The term “genome” generally refers to the genetic composition of an organism. In some instances, it may also refer to chromosomal, mitochondrial, bacterial, or other complement of DNA. Additionally what is referred to by those of ordinary skill in the related art as a genomic library may include a plurality of DNA, mRNA, EST, cDNA, or other type of sequence that represents the whole or a portion of a genome. For example, a genomic library may include collection of what are referred to as clones made from a set of randomly generated, sometimes overlapping DNA fragments representing all or part of a genome.

User 101 typically copies sequence information from sequences 316 and pastes this information into an HTML document accessible through NCBI's BLAST web pages 324 (as of November 2002, accessible at http://www.ncbi.nlm.nih.gov/BLAST/). This operation, which also may be time consuming and tedious if many sequences are involved, is represented by user-initiated batch BLAST request 322 of FIG. 3. BLAST is an acronym for Basic Local Alignment Search Tool, and, as is well known in the art, consists of similarity search programs that interrogate sequence databases for both protein and DNA using heuristic algorithms to seek local alignments. For example, user 101 may conduct a BLAST search using the “blastn” nucleotide sequence database. Results of this batch BLAST search, represented by similar nucleotide and/or protein sequence data 326, on occasion may not be available to user 101 for many minutes or even hours. User 101 may then initiate comparisons and evaluations 332, which may be conducted manually or using various software tools. User 101 may subsequently issue report 334 interpreting the findings of the searches and positing strategies and requirements for follow-on experiments.

Inputs to Genomic Portal 400 from User 101: The present invention may have preferred embodiments that include methods for providing genetic information over networks such as the Internet as described in U.S. patent application Ser. Nos. 10/063,559, 10/065,856; 10/065,868; 10/328,872; 10/328,818; and in U.S. Provisional Patent Application Serial Nos. 60/376,003; 60/394,574; and 60/403,381, which are all hereby incorporated by reference herein in their entireties for all purposes.

FIG. 4 is a functional block diagram showing an illustrative configuration by which user 101 may connect with genomic web portal 400. It will be understood that FIG. 4 is simplified and is illustrative only, and that many implementations and variations of the network and Internet connections shown in FIG. 4 will be evident to those of ordinary skill in the relevant art.

User 101 employs user computer 100 and analysis applications 199 as noted above, including generating and/or accessing some or all of files 212-217. As shown in FIG. 4, files 212-217 are maintained in this example on user database server 412 to which user computer 100 is coupled via network cable 480. Computers 100′, 100″, and computers of other users in a local or wide-area network including an Intranet, the Internet, or any other network may also be coupled to server 412 via cable 480. It will be understood that cable 400 is merely representative of any type of network connectivity, which may involve cables, transmitters, relay stations, network servers, and many other components not shown but evident to those of ordinary skill in the relevant art. Via user computer 100, user 101 may operate a web browser served by user-side Internet client 410 to communicate via Internet 499 with portal 400. Portal 400 may similarly be in communication over Internet 499 with other users and/or networks of users, as indicated by Internet clients 410′ and 410″.

As previously noted, the information provided by user 101 to portal 400 typically includes one or more “probe-set identifiers.” These probe-set identifiers typically come to the attention of user 101 as a result of experiments conducted on probe arrays. For example, user 101 may select probe-set identifiers that identify microarray probe sets capable of enabling detection of the expression of mRNA transcripts from corresponding genes or ESTs of particular interest. As is well known in the relevant art, an EST is a fragment of a gene sequence that may not be fully characterized, whereas a gene sequence generally is complete and fully characterized. The word “gene” is used generally herein to refer both to full size genes of known sequence and to computationally predicted genes. In some implementations, the specific sequences detected by the arrays that represent these genes or ESTs may be referred to as, “sequence information fragments (SIF's)” and may be recorded in a “SIF file,” as noted above with respect to the operations of LIMS 225. In particular implementations, a SIF is a portion of a consensus sequence that has been deemed to best represent the mRNA transcript from a given gene or EST. The consensus sequence may have been derived by comparing and clustering ESTs, and possibly also by comparing the ESTs to genomic sequence information. A SIF is a portion of the consensus sequence for which probes on the array are specifically designed. With respect to the operations of web portal 400, it is assumed with respect to some implementations that some microarray probe sets may be designed to detect the expression of genes based upon sequences of ESTs.

As was described above, the term “probe set” refers in some implementations to one or more probes from an array of probes on a microarray. For example, in an Affymetrix® GeneChip® probe array, in which probes are synthesized on a substrate, a probe set may consist of 30 or 40 probes, half of which typically are controls. These probes collectively, or in various combinations of some or all of them, are deemed to be indicative of a gene, EST, or protein. In a spotted probe array, one or more spots may similarly constitute a “probe set.”

The term “probe-set identifiers” is used broadly herein in that a number of types of such identifiers are possible and are intended to be included within the meaning of this term. One type of probe-set identifier is a name, number, or other symbol that is assigned for the purpose of identifying a probe set. This name, number, or symbol may be arbitrarily assigned to the probe set by, for example, the manufacturer of the probe array. A user may select this type of probe-set identifier by, for example, highlighting or typing the name. Another type of probe-set identifier as intended herein is a graphical representation of a probe set. For example, dots may be displayed on a scatter plot or other diagram wherein each dot represents a probe set. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. In another example, user 101 may select a probe-set identifier by selecting a row or column in a table or spreadsheet that correlates probe sets with accession numbers and other genomic information.

Yet another type of probe-set identifier, as that term is used herein, includes a nucleotide or amino acid sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIF. A user who specifies all or part of the 500-base sequence thus may be considered to have specified all or some of the corresponding probe sets.

In yet another example, a user may specify one or more SIF, gene, protein, or EST sequences for which there are no corresponding probe sets. The user requests an analysis of the specified sequences. User-service manager 522 (described below) assigns an identifier for a new probe set and this identifier, together with the sequence or sequences which are to be analyzed, are stored by database manager 512 in one or more databases. Manager 522 may submit probe sets for the corresponding SIF, gene, or EST and correlates the probe sets with the new probe-set identifiers. Further details regarding the processing and implementation of custom probe designs are provided in U.S. Provisional Patent Applications Nos. 60/301,298, and 60/265,103; and U.S. Patent Applications Nos. 09/824,931, and 10/065,868; each of which is hereby incorporated by reference herein in its entirety for all purposes.

A further example of a probe-set identifier is an accession number of a gene or EST. Gene and EST accession numbers are publicly available. A probe set may therefore be identified by the accession number or numbers of one or more ESTs and/or genes corresponding to the probe set. The correspondence between a probe set and ESTs or genes may be maintained in a suitable database, such as that accessed by database application 230 or local library databases 516, from which the correspondence may be provided to the user. Similarly, gene fragments or sequences other than ESTs may be mapped (e.g., by reference to a suitable database) to corresponding genes or ESTs for the purpose of using their publicly available accession numbers as probe-set identifiers. For example, a user may be interested in genomic information related to a particular SIF that is derived from EST-1 and EST-2. The user may be provided with the correspondence between that SIF (or part or all of the sequence of the SIF) and EST-1 or EST-2, or both. To obtain genomic data or analyze the sequence related to the SIF, or a partial sequence of it, the user may select the accession numbers of EST-1, EST-2, or both.

Additional examples of probe-set identifiers include one or more terms that may be associated with the annotation of one or more gene or EST sequences, where the gene or EST sequences may be associated with one or more probe sets. For convenience, such terms may hereafter be referred to as “annotation terms” and will be understood to potentially include, in various implementations, one or more words, graphical elements, characters, or other representational forms that provide information that typically is biologically relevant to or related to the gene or EST sequence. Associations between the probe-set identifier terms and gene or EST sequences may be stored in a database such as Probe-set ID to sequence database 511, local genomic database 518, or they may be transferred from remote databases 402. Examples of such terms associated with annotations include those of molecular function (e.g. transcription initiation), cellular location (e.g. nuclear membrane), biological process (e.g. immune response), tissue type (e.g. kidney), or other annotation terms known to those in the relevant art.

To provide a further specific example, user 101 may input the illustrative annotation term “tumor suppression.” A large number of genes or ESTs are known to be involved with this biological process. For example, a gene known as p53 is involved with tumor suppression, and this information is stored in one or more of the databases accessible from database server 410. Portal 400 provides to user 101 a list of probe-set identifiers that includes the one or more probe-set identifiers associated with gene p53. The list of probe-set identifiers may be provided to the user in one of numerous possible formats. For example, the format may include a table comprising all the probe sets associated with all the genes or ESTs associated with “tumor suppression.” Alternatively, the format may separate the probe sets related to each gene or EST into its own table.

Genomic web portal 400: Genomic web portal 400 provides to user 101 data related to one or more genes, ESTs, or proteins. Feature elements that make up a gene include: exons, 5′ and 3′ untranslated regions, coding regions, start and stop codons, introns, 5′ transcriptional control elements, 3′ polyadenylation signals, splice site boundaries, and protein-based annotations of the coding regions.

In some implementations, what those of ordinary skill in the related art refer to as alternative splice variants may include groups of MRNA, EST, or protein sequences derived from the same genomic region. For example, a group of alternative splice variants could include two or more mRNA sequences each sharing a minimum level of sequence identity that may for instance include a minimum of 50 bases that are common to the group in composition and relative position. In the present example, each alternative splice variant in the group may have been “spliced” from a common primary transcript, and differ from one another in exon composition and arrangement. Additionally, in the present context alternative splice variants may also be conceptualized as a plurality of different nucleotide sequences that are transcribed from the same gene and upon translation yield peptide or protein sequences having a minimal number of common amino acids, arranged in the same order, wherein the minimal number of amino acids may be at least 15 amino acids.

A molecular apparatus commonly referred to as the “splicesome” performs a process referred to as RNA processing after a gene has been transcribed into a primary RNA transcript. The splicesome cleaves the primary RNA transcript at specific locations such as what are referred to in the art as intron/exon boundaries. After cleavage, the splicesome arranges the cleaved sequence and splices the sequence together, generally leaving out the intron sequences and possibly leaving out one or more exon sequences. The splicesome may produce alternative splice variants by altering the number, arrangement, and/or content (i.e., by splicing one or more intron/exon portions) of exons. Thus, alternative splice variants could also include the arrangement of partial sequence from exons that, for instance, may include alternative 3′ and 5′ splice sites. Additionally, as is well known to those of ordinary skill in the art, alternative splice variants may be produced not only by alternative splicing but also by other methods, for example, alternative promoter site choice and alternative polyadenylation sites. Those of ordinary skill in the related art will appreciate that approximately a third to over half of all human genes produce multiple alternative splice variants (E. S. Lander, et al., “Initial sequencing and analysis of the human genome,” Nature, vol. 409, pp. 860-921, 2001; A. A. Mironov, J. W. Fickett, and M. S. Gelfand, “Frequent alternative splicing of human genes,” Genome Res, vol. 9, pp. 1288-93, 1999), which are both hereby incorporated by reference herein in their entireties. Each alternative splice variant could have different expression patterns and function. It is also generally appreciated that alternative splicing is an important regulatory mechanism in higher eukaryotes. For example, a gene could include three exons that for the purposes of illustration may be referred to as exon 1, exon 2, and exon 3. In the present example, a plurality of alternative splice variants from that gene are possible that could include an EST composed of exons 1, 2, and 3; another EST composed of exons 1, and 2; or an EST composed of exons 1 and 3 or yet another EST composed of exons 2 and 3.

Typically, each gene or EST has at least one corresponding probe set that is identified by a probe-set identifier that, as just noted, may be a number, name, accession number, symbol, graphical representation (e.g., dot or highlighted tabular entry), and/or nucleotide sequence, as illustrative and non-limiting examples. The corresponding probe sets are capable of enabling detection of the expression of their corresponding gene or alternative splice variant. In some embodiments a probe set designed to recognize the mRNA expression of a gene may identify one or more alternative splice variants. In some cases a plurality of probe sets may be capable of identifying a specific alternative splice variant.

In some embodiments, probe sets are designed to identify specific alternative splice variants. For example, a probe set may consist of probes designed to interrogate the exons of a particular alternative splice variant as well as junction probes designed to interrogate the region where two specific exons are predicted to be joined together. The junction probe may interrogate, for instance, the sequence of the 3′ end of exon 1 and the 5′ end of exon 3. In the present example, an alternative splice variant mRNA that comprises exons 1 and 3 will hybridize to the exon probes and, if the splice variant is joined in the correct orientation, it will also hybridize to the one or more junction probes. Additional examples of alternative splice variant probe sets and probe arrays are described in U.S. patent application Ser. Nos. 09/697,877, and 10/384,275, each of which is hereby incorporated by reference herein in its entirety for all purposes.

In response to a user selection of one or more probe-set identifiers, portal 400 provides user 101 with one or more of genomic, EST, protein, or annotation information. This information may be helpful to user 101 in analyzing the results of experiments and in designing or implementing follow-up experiments.

FIG. 5 is a functional block diagram of one of many possible embodiments of portal 400. In this example, portal 400 has hardware components including three computer platforms: database server 510, Internet server 530, and application server 520. Various functional elements of portal 400, such as database manager 512, input and output managers 532 and 534, and user-service manager 522, carry out their operations on these computer platforms. That is, in a typical implementation, the functions of managers 512, 532, 534, and 522 are carried out by the execution of software applications on and across the computer platforms represented by servers 510, 530, and 520. Portal 400 is described first with respect to its computer platforms, and then with respect to its functional elements.

Each of servers 510, 520 and 530 may be any type of known computer platform or a type to be developed in the future, although they typically will be of a class of computer commonly referred to as servers. However, they may also be a main frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Sun Solaris, Linux, OS/400, Compaq Tru64 Unix, SGI IRIX, Siemens Reliant Unix, and others.

There may be significant advantages to carrying out the functions of portal 400 on multiple computer platforms in this manner, such as lower costs of deployment, database switching, or changes to enterprise applications, and/or more effective firewalls. Other configurations, however, are possible. For example, as is well known to those of ordinary skill in the relevant art, so-called two-tier or N-tier architectures are possible rather than the three-tier server-side component architecture represented by FIG. 5. See, for example, E. Roman, Mastering Enterprise JavaBeans™ and the Java™2 Platform (Wiley & Sons, Inc., NY, 1999) and J. Schneider and R. Arora, Using Enterprise Java™ (Que Corporation, Indianapolis, 1997), both of which are hereby incorporated by reference in their entireties for all purposes.

It will be understood that many hardware and associated software or firmware components that may be implemented in a server-side architecture for Internet commerce are not shown in FIG. 5. Components to implement one or more firewalls to protect data and applications, uninterruptible power supplies, LAN switches, web-server routing software, and many other components are not shown. Similarly, a variety of computer components customarily included in server-class computing platforms, as well as other types of computers, will be understood to be included but are not shown. These components include, for example, processors, memory units, input/output devices, buses, and other components noted above with respect to user computer 100. Those of ordinary skill in the art will readily appreciate how these and other conventional components may be implemented.

The functional elements of portal 400 also may be implemented in accordance with a variety of software facilitators and platforms (although it is not precluded that some or all of the functions of portal 400 may also be implemented in hardware or firmware). Among the various commercial products available for implementing e-commerce web portals are BEA WebLogic from BEA Systems, which is a so-called “middleware” application. This and other middleware applications are sometimes referred to as “application servers,” but are not to be confused with application server 520, which is a computer. The function of these middleware applications generally is to assist other software components (such as managers 512, 522, or 532) to share resources and coordinate activities. The goals include making it easier to write, maintain, and change the software components; to avoid data bottlenecks; and prevent or recover from system failures. Thus, these middleware applications may provide load-balancing, fail-over, and fault tolerance, all of which features will be appreciated by those of ordinary skill in the relevant art.

Other development products, such as the Java™ 2 platform from Sun Microsystems, Inc. may be employed in portal 400 to provide suites of applications programming interfaces (API's) that, among other things, enhance the implementation of scalable and secure components. The platform known as J2EE (Java™2, Enterprise Edition), is configured for use with Enterprise JavaBeans™, both from Sun Microsystems. Enterprise JavaBeans™ generally facilitates the construction of server-side components using distributed object applications written in the Java™ language. Thus, in one implementation, the functional elements of portal 400 may be written in Java and implemented using J2EE and Enterprise JavaBeans™. Various other software development approaches or architectures may be used to implement the functional elements of portal 400 and their interconnection, as will be appreciated by those of ordinary skill in the art.

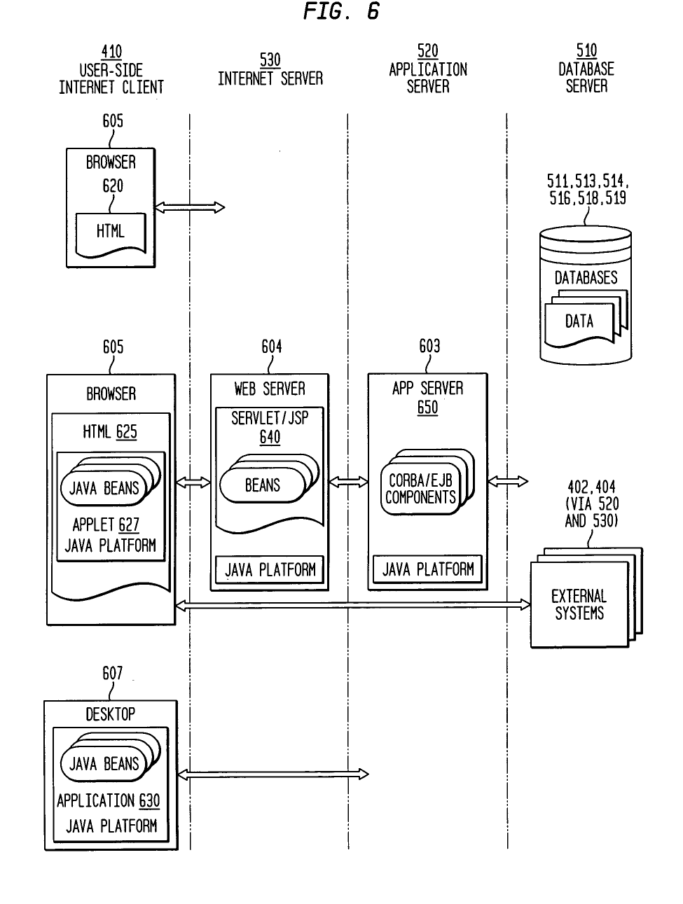

One implementation of these platforms and components is shown in FIG. 6. FIG. 6 is a simplified graphical representation of illustrative interactions between user-side internet client 410 on the user side and input and output managers 532 and 534 of Internet server 530 on the portal side, as well as communications among the three tiers (servers 510, 520, and 530) of portal 400. Browser 605 on client 410 sends and receives HTML documents 620 to and from server 530. HTML document 625 includes applet 627. Browser 605, running on user computer 100, provides a run-time container for applet 627. Functions of managers 532 and 534 on server 530, such as the performance of GUI operations, may be implemented by servlet and/or JSP 640 operating with a Java™ platform. A servlet engine executing on server 530 provides a runtime container for servlet 640. JSP (Java Server Pages) from Sun Microsystems, Inc. is a script-like environment for GUI operations; an alternative is ASP (Active Server Pages) from the Microsoft Corporation. App server 650 is the middleware product referred to above, and executes on application server 520. EJB (Enterprise JavaBeans™ is a standard that defines an architecture for enterprise beans, which are application components. CORBA (Common Object Request Broker Architecture) similarly is a standard for distributed object systems, i.e., the CORBA standards are implemented by CORBA-compliant products such as Java™ IDL. An example of an EJB-compliant product is WebLogic, referred to above. Further details of the implementation of standards, platforms, components, and other elements for an Internet portal and its communications with clients, are well known to those skilled in the relevant art.

As noted, one of the functional elements of portal 400 is input manager 532. Manager 532 receives a set, i.e., one or more, of probe-set identifiers from user 101 over Internet 499. Manager 532 processes and forwards this information to user-service manager 522. These functions are performed in accordance with known techniques common to the operation of Internet servers, also commonly referred to in similar contexts as presentation servers. Another of the functional elements of portal 400 is output manager 534. Manager 534 provides information assembled by user-service manager 522 to user 101 over Internet 499, also in accordance with those known techniques, aspects of which were described above in relation to FIG. 6. The information assembled by manager 522 is represented in FIG. 5 as data 524, labeled “integrated genomic and/or product web pages responsive to user request.” The data is integrated in the sense, among other things, that it is based, at least in part, on the specification by user 101 of probe-set identifiers and thus has common relationships to the genes and/or ESTs, or proteins corresponding to those identifiers. The presentation by manager 534 of data 524 may be implemented in accordance with a variety of known techniques. As some examples, data 524 may include HTML or XML documents, email or other files, or data in other forms. The data may include Internet URL addresses so that user 101 may retrieve additional HTML, XML, or other documents or data from remote sources.

Portal 400 further includes database manager 512. In the illustrated embodiment, database manager 512 coordinates the storage, maintenance, supplementation, and all other transactions from or to any of local databases 511, 515, 516, 518 and 519. Manager 512 may undertake these functions in cooperation with appropriate database applications such as the Oracle® 8.0.5 database management system.

In some implementations, manager 512 periodically updates local genomic database 518. The data updated in database 518 includes data related to genes, ESTs, or proteins that correspond with one or more probe sets. The probe sets may be those used or designed for use on any microarray product, and/or that are expected or calculated to be used in microarray products of any manufacturer or researcher. For example, the probe sets may include all probe sets synthesized on the line of stocked GeneChip® probe arrays from Affymetrix, Inc., including its Arabidopsis Genome Array, C. elegans Genome Array, Drosophila Genome Array, E. coli Genome Array, Human Genome Focus Array, Human Genome U133 Set, Human Genome U95 Set, Mouse Expression Set 430, Murine Genome U74v2 Set, P. aeruginosa Genome Array, Rat Expression Set 230, Rat Genome U34 Set, Rat Neurobiology U34 Array, Rat Toxicology U34 Array, Test3 Array, Yeast Genome S98 Array, CYP450 Array, GenFlex Tag Array, HuSNP Probe Array and p53 Probe Array. The probe sets may also include those synthesized on alternative splice arrays or custom arrays for user 101 or others. However, the data updated in database 518 need not be so limited. Rather, it may relate, e.g., to any number of genes, ESTs, or proteins. Types of data that may be stored in database 518 are described below in relation to the operations of manager 522 in directing the periodic collection of this data from remote sources providing the locally maintained data in database 518 to users.

Database 516 includes data of a type referred to above in relation to database application 230, i.e., data that associates probe sets with their corresponding gene or EST and their identifiers. Database 516 may also include SIF's, and other library data. User-service manager 522 may provide database manager 512 from time to time with update information regarding library and other data. In some cases, this update information will be provided by the owners or managers of proprietary information, although this information may also be made available publicly, as on a web site, for uploading.

Database 511 includes information relating probe-set identifiers to the sequences of the probes. This information may be provided by the manufacturer of the probes, the researchers who devise probes for spotted arrays or other custom arrays, or others. Moreover, the application of portal 400 is not limited to probes arranged in arrays. As noted, probes may be immobilized on or in beads, optical fibers, or other substrates or media. Thus, database 511 may also include information regarding the sequences of these probes.

Database 519 includes information about users and their accounts for doing business with or through portal 400. Any of a variety of account information, such as current queries and orders, past queries and orders, and so on, may be obtained from users, all as will be readily apparent to those of ordinary skill in the art. Also, information related to users may be developed by recording and/or analyzing the interactions of users with portal 400, in accordance with known techniques used in e-commerce. For example, user-service manager 522 may take note of users' areas of genomic interest, their query activities, the frequency of their accessing of various services, and so on, and provide this information to database manager 512 for storage or update in database 519.

Another functional element of portal 400 is user-service manager 522. Among other functions, manager 522 may periodically cause database manager 512 to update local genomic database 518 from various sources, such as remote databases 402. For example, according to any chronological schedule (e.g., daily, weekly, etc.), or need-driven schedule (e.g., in response to a user making an authorized request for updated information), manager 522 may, in accordance with known techniques, initiate searches of remote databases 402 by formulating appropriate queries, addressed to the URL's of the various databases 402, or by other conventional techniques for conducting data searches and/or retrieving data or documents over the Internet. These search queries and corresponding addresses may be provided in a known manner to output manager 534 for presentation to databases 402. Input manager 532 receives replies to the queries and provides them to manager 522, which then provides them to database manager 512 for updating of database 518, all in accordance with any of a variety of known techniques for managing information flow to, from, and within an Internet site.

Portal application manager 526 manages the administrative aspects of portal 400, possibly with the assistance of a middleware product such as an applications server product. One of these administrative tasks may be the issuance of periodic instructions to manager 522 to initiate the periodic updating of database 518 just described. Alternatively, manager 522 may self-initiate this task. It is not required that all data in database 518 be updated according to the same periodic schedule. Rather, it may be typical for different types of data and/or data from different sources to be updated according to different schedules. Moreover, these schedules may be changed, and need not be according to a consistent schedule. That is, for example, updating for particular data may occur after a day, then again after 2 days, then at a different period that may continue to vary. Numerous factors may influence the determination by manager 526 or manager 522 to maintain or vary these periods, such as the response time from various remote databases 402, the value and/or timeliness of the information in those databases, cost considerations related to accessing or licensing the databases, the quantity of information that must be accessed, and so on.

In some implementations, manager 522 constructs from data in local genomic database 518 a set of data related to genes, ESTs, or proteins corresponding to the set of probe-set identifiers selected by user 101. The user selection may be forwarded to manager 522 by input manager 532 in accordance with known techniques. Manager 522, also in accordance with known techniques, obtains the data from database 518 by forming appropriate queries, such as in one of the varieties of SQL language, based on the user selection. Manager 522 then forwards the queries to database manager 512 for execution against database 518. Other techniques for extracting information from database 518 may be used in alternative implementations.

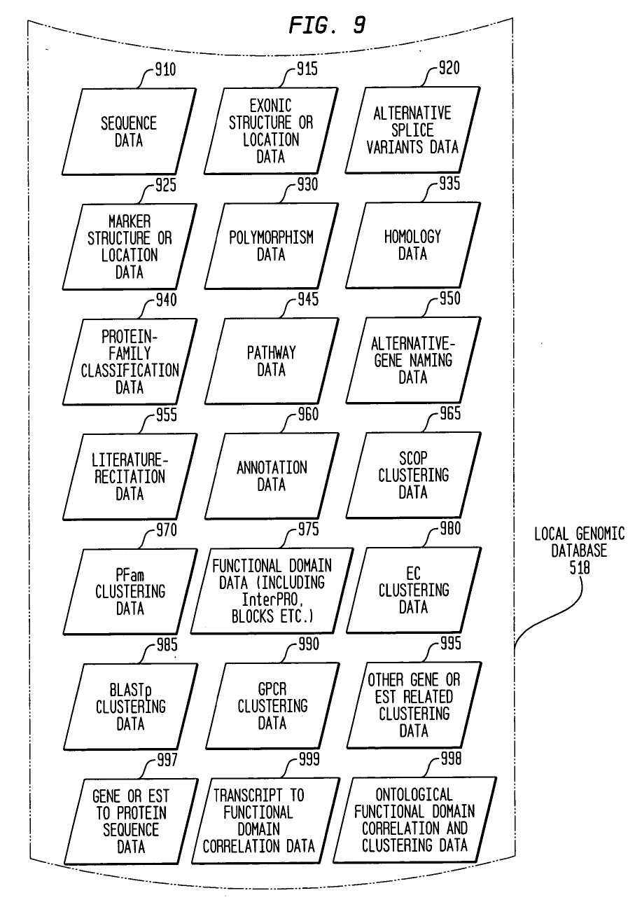

As noted, various types of data may be accessed from remote databases 402 and maintained in local genomic database 518. Examples are illustrated in FIG. 9 that include sequence data 910, exonic structure or location data 915, alternative splice variants data 920, marker structure or location data 925, polymorphism data 930, homology data 935, protein-family classification data 940, pathway data 945, alternative-gene naming data 950, literature-recitation data 955, annotation data 960, functional domain data 975, gene or EST to protein sequence data 997, transcript to functional domain correlation data 999 and various clustering data, including ontological functional domain correlation and clustering data 998, SCOP clustering data 965, PFam clustering data 970, EC clustering data 980, BLASTp clustering data 985 and other gene or EST related clustering data 995. Many other examples are possible. Also, genomic data not currently available but that becomes available in the future may be accessed and locally maintained as described herein. Examples of remote databases 402 currently suitable for accessing in the manner described include GenBank, GenBank New, SwissProt, GenPept, DB EST, Unigene, PIR, Prosite, Pfam, Prodom, eMotif, Blocks, PDB, PDBfinder, EC Enzyme, Kegg Pathway, Kegg Ligand, OMIM, OMIM Map, OMIM Allele, DB SNP, Gene Ontology, SeqStore®, PubMed, SWALL, InterPro, and LocusLink. Hundreds of other databases currently exist that are suitable, any many more will be developed in the future that may be included as aspects of databases 402, and thus this list is merely illustrative.

Moreover, local genomic database 518 may also be supplemented with data obtained or deduced (by user-service manager 522) from other of the local databases serviced by database manager 512. Also, in some implementations, data may be retrieved from one or more of remote databases 402 in real time with respect to a user request rather than from locally maintained database 518.

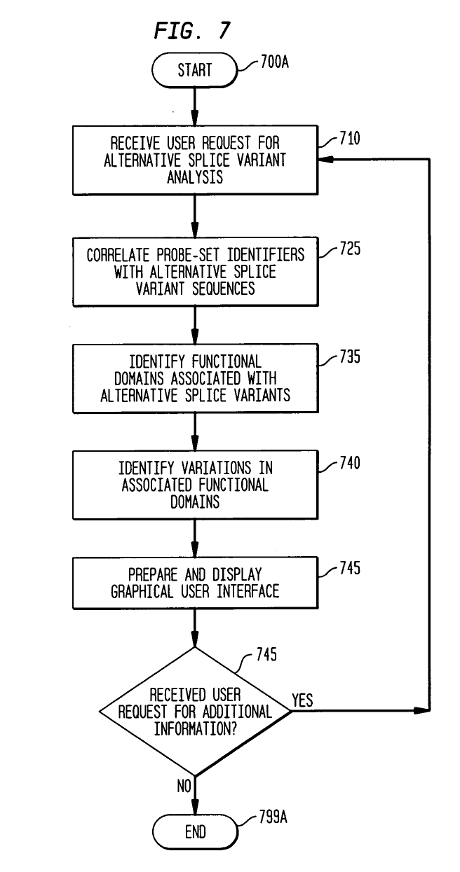

More specific examples are now provided of how user service manager 522 may receive and respond to requests from user 101 for genomic, EST, protein, or annotation information, as well as for product information and/or ordering. These examples are described in relation to FIGS. 7 through 12.

FIG. 7 is a flow chart representing one of the many possible illustrative methods by which portal 400 may respond to a user's request for genomic information related to analysis of alternative splice variants. In accordance with step 710 of this example, input manager 532 receives from client 410 over Internet 499 a request by user 101. This request may, for instance, include an HTML, XML, or text document (e.g., tab delimited *.txt document) that includes user 101's selection of certain probe-set identifiers. As noted, the probe-set identifiers may be a number, name, accession number, symbol, graphical representation, or nucleotide, protein or other biological sequence, as non-limiting examples. In some cases, user 101 may make this selection by employing one or more of analysis applications 199A to select probe-set identifiers (e.g., by drawing a loop around dots, selecting portions of a graph or spreadsheet, or other methods as noted above) and then activating communication with portal 400 by any of a variety of known techniques such as right-clicking a mouse. The request may also, in accordance with any of a variety of known techniques, specify that user 101 is interested in genomic data and/or analysis of data, as well as details regarding the type of data and/or the type of analysis that is desired. For instance, user 101 may select genes, alternative splice variants, proteins, suitable analysis methods and so on from pull-down menus. Manager 532 provides user 101's request to user service manager 522, as described above.

In accordance with step 725, user-service manager 522 in one implementation formulates an appropriate query (using, for example, a version of the SQL language) for correlating probe-set identifiers with corresponding genes, ESTs, or proteins. Gene or EST determiner 820 is the functional element of manager 522 that executes this task in the illustrated example. Determiner 820 forwards the query to database manager 512. If the probe-set identifiers provided by user 101 include sequence information, then the query may seek to determine the existence of one or more corresponding probe sets, consisting of probes, from database 511, and/or from SIF information in database 516. Determiner 820 may further correlate the identity of the one or more probe sets having a corresponding (e.g., similar in biological significance) sequence with the probe-set identifiers.

In some implementations, the probe sequences determined by determiner 820 may be used as an identifier for an unknown, e.g., as yet not provided, probe-set. Also, in some implementations, the probe-set identifiers could include one or more terms (e.g. referring to annotation information such as “tumor suppressor”). In either case, user service manager 522 may identify the genes, ESTs, or proteins from database 518, where annotation information is stored with the corresponding genes, ESTs, or proteins. If the probe-set identifiers include names or numbers (e.g., accession numbers), then the query may seek the identity of the probe sets from database 516 that, as noted, includes data that associates names, numbers, and other probe-set identifiers with corresponding genes or ESTs. User 101 may also have locally employed database application 230 to obtain this information, and include this information in the information request in accordance with known techniques. In this case, step 725 need not be performed.

In some embodiments, determiner 820 may perform methods for evaluating the presence of alternative splice variants in one or more experiments from an input set of one or more probe-set identifiers and associated hybridization intensities from the one or more experiments. In one implementation, determiner 820 may receive an input set of probe-set identifiers and associated hybridization intensities derived from the results of probe array experiments. Determiner 820 performs methods of a kind typically referred to by those of ordinary skill in the relevant art as “model fitting” to evaluate the probe-set identifiers and associated hybridization intensities for alternative splice variants. For example, determiner 820 receives a set of probe-set identifiers and the hybridization intensities associated with each probe-set identifier from a user via input manager 532. Determiner 820 of this implementation formulates a query to database manager 512 to retrieve data related to alternative splice variant sequences and protein functional domains based, at least in part, upon the input probe-set identifiers. The data related to alternative splice variant sequences and functional domains could for instance include data stored in transcript to functional domain correlation data 999, exon structure or location data 915, protein-family classification data 940, homology data 935, functional domain data 975, gene or EST to protein sequence data 997, ontological functional domain correlation and clustering data 998 or alternative splice variants data 920. Determiner 820 fits the probe-set identifiers and associated hybridization data to models of known alternative splice variant sequences using, for example, an iterative model-fitting algorithm. For instance, it may be illustratively assumed that a pattern of hybridization data strongly indicates the presence of exons 1 and 3 because probe sets representing those exons have been detected with high intensity values. These data may be taken to indicate that one or more splice variants that include exons 1 and 3 are present. The intensity values related to exons 2 and 4 may, of course, also be relevant to this determination and may change the determination based on the overall best fit of the data. In the present example, each iteration of the algorithm improves the quality of the fit of the data to the known models. One such model, for example, is a linear model that assumes a normal distribution of variables. It will be apparent to those of ordinary skill in the related art that a variety of different models could be implemented that may also include a variety of assumptions regarding the distribution of variables.

The fit may, in some implementations, be verified using the alternative splice variants and functional domain data listed above. For example, determiner 820 may verify a fit of the probe-set identifier and hybridization intensity data to a model of a particular splice variant by comparing the known function of that splice variant (assuming that there is a known function) to the collective properties of the combined functional domains identified by the data. For instance, the data may identify one or more DNA binding domains that relate to promoter region of a specific gene. Determiner 820 may have fit the data to a model of an alternative splice variant that has a known function as a transcription factor of the same gene. In the present example, determiner 820 verifies that there is an accurate fit of the data to the model. Additional examples of model fitting and evaluation of alternative splice variants are provided in U.S. patent application Ser. No. 09/697,877 in U.S. Provisional Patent Applications Nos. 60/362,315, 60/362,524, 60/362,454, 60/362,455, 60/362,399, 60/375,351, 60/384,552, 60/398,958, and 60/422,220, titled “METHOD OF ANALYZING ALTERNATIVE SPLICING”, filed Oct. 29, 2002, each of which is hereby incorporated by reference herein in its entirety for all purposes.

In the same or alternative implementation, a user may input a set of one or more probe-set identifiers for the purpose of identifying associated alternative splice variants so that the user may design an experiment that may be intended, for example, to further analyze transcript or splice variants. For example, determiner 820 may formulate a query to database manager 512 to determine alternative splice variants that are known to correspond to the input set of one or more probe-set identifiers provided by the user. Manager 512 retrieves the alternative splice variant data from alternative splice variants data 920 of local genomic database 518, or from other databases located locally or remotely. Determiner 820 then forwards retrieved data to correlator 830.

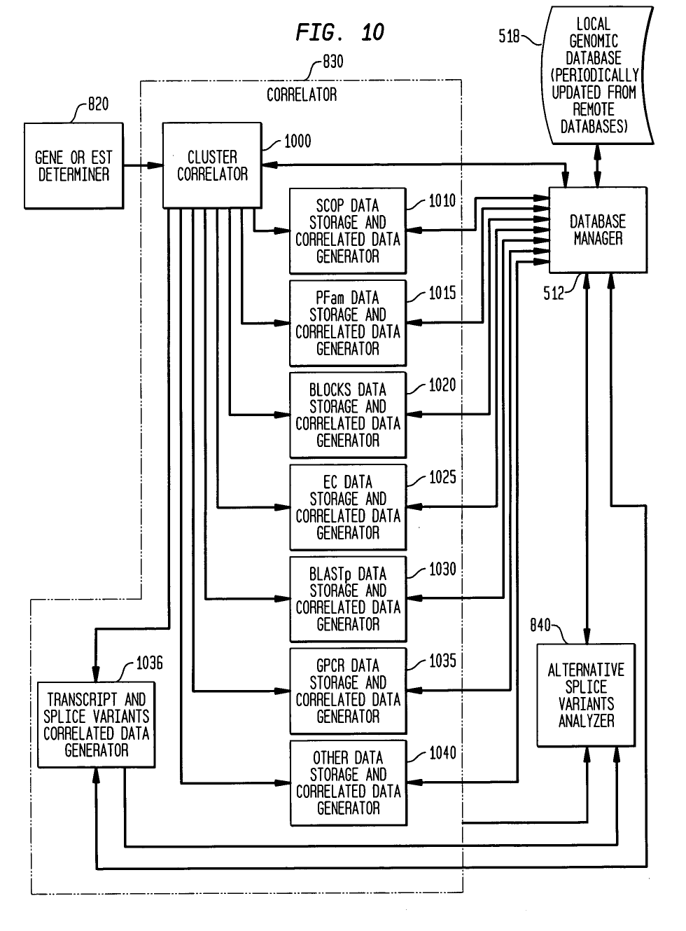

An implementation of correlator 830 is illustrated in FIG. 10, wherein cluster correlator 1000 receives from gene or EST determiner 820 a nucleotide sequence that may or may not correspond to a probe set. Cluster correlator may correlate the nucleotide sequence via database manager 512 with a corresponding protein sequence found in gene or EST to protein sequence data 997, as is illustrated in FIG. 9, or alternatively, correlator 1000 may translate the nucleotide sequence into a protein sequence by methods known to those of ordinary skill in the art. Cluster correlator 1000 then sends the protein sequence to data storage and correlated data generators 1010, 1015, 1020, 1025, 1030, 1035, 1036 and 1040. The data storage and correlated data generators correspond to databases, now available or that may be developed in the future, that contain information regarding associated protein family, pathway, network, complex, transcript and/or splice variants, and/or other protein annotation information. Such databases include but are not limited to, SCOP, PFam, BLOCKS, eMotif, EC, InterPRO and GPCR, which are known to those in the art as databases that contain annotation information. Such clusters of data may be stored in local genomic database 518 as illustrated in FIG. 9 as clustering data including ontological functional domain correlation and clustering data 998, SCOP clustering data 965, PFam clustering data 970, EC clustering data 980, BLASTp clustering data 985, GPCR clustering data 990 and other gene or EST related clustering data 995. The databases used in this example are for illustration only, and those of ordinary skill in the art know that many other examples are possible.

The data storage and correlated data generators use methods, known to those in the art as clustering methods, to determine sequence or structural similarity and alignments with similar protein sequences and/or structures. There are numerous types of clustering methods used for these purposes, for example what is commonly known as BLASTp represented in FIGS. 9 and 10 as BLASTp clustering data 985 and BLASTp data storage and correlated data generator 1030 respectively.

Another example is commonly referred to as the Hidden Markov Model (referred to hereafter as HMM). HMM's are pattern matching algorithms that use a training set of data to “learn” the patterns contained in that training set of data. One implementation is the so-called GRAPA set of HMM's that are trained to be specific to families of proteins where each family has its own HMM trained to its characteristic pattern (GPCR-GRAPA-LIB-a refined library of hidden Markov Models for annotating GPCRs, Shigeta R, et.al., Bioinformatics Mar. 22, 2003; 19(5):667-8, incorporated herein by reference in its entirety for all purposes.) A trained HMM can then analyze a sequence and return a score that corresponds to how well the sequence matches the pattern. In one illustrative implementation, a threshold value is assigned so that a score above the threshold is considered to be a member of the family and a score below is not. The data storage and correlated data generators of this implementation then generate what is commonly referred to as a pairwise alignment between the query sequence and the family consensus sequence, and correlate annotation data corresponding to the family.

An additional implementation of correlator 830 includes receiving data regarding alternative splice variants from determiner 820. Data so received is illustratively shown as received and processed by alternative splice variants correlated data generator 1036. Generator 1036 formulates a query to database manger 512 to find alternative splice variants, protein functional domain and annotation information, based at least in part upon data regarding alternative splice variants. In some implementations, for example, generator 1036 in this manner retrieves information that includes genomic structural domains, functional domains, translation frame and annotations for each alternative splice variant contained in data regarding alternative splice variants received from determiner 820. Generator 1036 may forward the received data, genomic structural domains and protein functional domains, to database manager 512 for storage in one or more databases, as well as to alternative splice variants analyzer 840 for further processing and/or incorporation into one or more graphical user interfaces for presentation to a user.

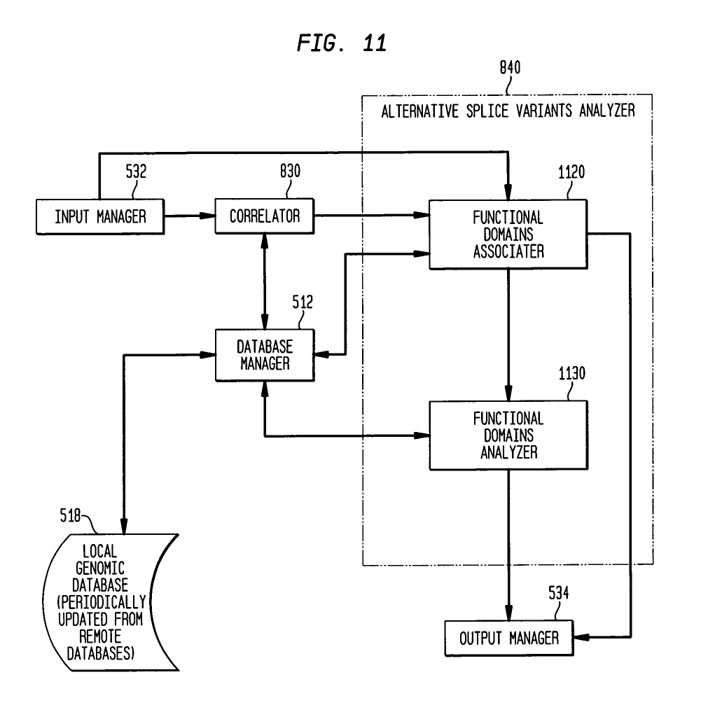

Some embodiments of portal 400 may include alternative splice variants analyzer 840, described in detail with respect to FIG. 11 below that receives alternative splice variant sequences from correlator 830 and/or from input manger 532. Analyzer 840 may identify functional differences between alternative splice variants such as, for instance, variation in exon composition and arrangement. Such functional differences may be based, at least in part, upon what are referred to by those of ordinary skill in the related art as “functional domains” or “motifs”, defined by the exon composition and arrangement of the particular variants. As is known to those of ordinary skill in the relevant art, proteins often include functional domains, modules or motifs that have distinct functional characteristics. Furthermore, it may also be noted that the term “functional domain” is used broadly and non-restrictively in the present context and generally refers to annotation data related to the one or more “functional domains” including, but not limited to, name of the domain, other alphanumeric domain identifiers, nucleotide and/or protein sequences known to be associated with the functional domain and so on. It will also be appreciated by those of ordinary skill in the related art that the exon identity and/or the functional domains may depend upon what is referred to in the art as the translation or reading frame.

Analyzer 840 may present the identified functional differences in one or more GUIs, such as GUI 1200, or alternatively forward the related information to output manager 534 for presentation in GUI 1200 and/or storage in one or more databases.

Additionally, analyzer 840 may determine the putative function of proteins produced by each alternative splice variant based, at least in part, upon the combination of one or more functional domains identified. For example, analyzer 840 may determine the putative function by relating the combination of the identified functional domains to one or more known proteins that have similar combinations of functional domains. In the present example, the alternative splice variant may be identified as a cell surface receptor by the combination of what is referred to as seven transmembrane regions and one or more receptor domains which may be partially composed of the transmembrane segments.

FIG. 11 is a functional block diagram of one embodiment of alternative splice variants analyzer 840 for functional analysis of alternative splice variants. Analyzer 840 includes functional domains associater 1120 and functional domains analyzer 1130. Functional domains associater 1120 may receive alternative splice variant sequences directly from input manger 532 as provided by the user 101 and/or after processing by correlator 830 if user 101 provides data in a form other than as alternative splice variant sequences. In some implementations, user 101 may provide one or more probe set identifiers and associated intensity values from one or more biological experiments, where the probe set identifiers may be provided to correlator 830 for correlation with one or more alternative splice variant sequences. For example, if the probe set identifiers provided by user 101 include gene names or accession numbers, correlator 830 may correlate the gene names or accession numbers with appropriate alternative splice variant sequences. The alternative splice variant sequences may be provided by correlator 830 to associater 1120. In the same or other implementations user 101 may also provide one or more sequences comprising one or more regions of a genome and/or one or more of overlapping EST or RNA sequences which may be correlated with known alternative transcripts. Additionally, a set of alternative splice variant sequences may be deduced from the one or more sequences provided by user 101.

Functional domains associater 1120 performs queries to one or more databases such as database 518, via database manger 512, based, at least in part, upon the plurality of alternative splice variant sequences received from correlator 830 and/or manager 532. Associater 1120 may determine one or more functional domains associated with one or more regions of the alternative splice variant sequences. Associater 1120 may query database 518 for transcript to functional domain correlation data 999 and correlate the alternative splice variant sequences to the sequences associated with one or more functional domains. For example, various portions or regions of alternative splice variant sequences may be correlated with one or more functional domains by searching the data 999 for sequences same as or similar to the alternative splice variant sequences using one or more sequence similarity searching techniques well known to those of skill in the art, such as, but not limited to, regular expression search and so on. Additionally, the one or more sequence similarity searching techniques may include techniques employing one or more measures of similarity that may be used as the basis of correlation. For example, as is well known to those of skill in the art, BLAST searching may be used to compare two sequences and a measure of similarity may be calculated, including, a numerical similarity score. Alternatively, other sequence similarity searching techniques, well known to those of skill in the art, may be employed.

Data 999 may employ a data model suitable for biological sequence analysis such as in the illustrated implementation of determining functional domains associated with alternative splice variant sequences. The term “data model”, as used herein, generally refers to a representation of one or more elements within a selected type of data that, for instance, may be implemented by a computer database to catalog and store data in a useable fashion. As those of ordinary skill in the related art will appreciate, the data model may include what is referred to as a hierarchical, network, object oriented, object-relational, entity-relationship, or other type of data model. Additionally, a data model may be represented using the Unified Modeling Language (commonly referred to as UML), Data Manipulation Language (commonly referred to as DML), or other type of language known to those of ordinary skill in the related art.

Some implementations of data models used for biological sequence analysis may utilize BioPerl, BioJava, BioPython, or other types of tools or modules known to those of ordinary skill in the related art to perform various functions required by the data model. For example, a data model may include a generalized and unified data model for representing biological sequence and their relationships that may be implemented in what is known to those in the art as an object oriented design philosophy. Annotations are included in what are commonly referred to as objects of the data model as compared, for example, to conventional schemes in which annotations may be associated with sequence information. In the present example, the data model may incorporate annotations directly in the data objects so that the annotation for a sequence may be found in one or more data objects representing a chromosome, contiguous fragment or sequence, bacterial artificial chromosome, or other sequence entity.

A data model may offer many advantages including, user flexibility to manipulate sequence information for particular needs and efficiency in terms of both memory and computational time. Methods that may be used for generating and representing data 999 are described in U.S. Provisional Patent application Serial No. 60/375,907 and United States Patent Application, Attorney Docket No. 3471.1, titled “SYSTEM, METHOD, AND COMPUTER PROGRAM PRODUCT FOR THE REPRESENTATION OF BIOLOGICAL SEQUENCE DATA”, both of which are incorporated by reference above. Additionally, associater 1120 may determine the functional domains by analyzing alternative splice variant sequences using what is known to those of skill in the art as homology modeling, or other methods, such as, by employing HMMs as described above.

Now returning to FIG. 11, associater 1120 may determine the putative function of proteins produced by each alternative splice variant based upon the identified functional domains and ontological functional domain correlation and clustering data 998 (details regarding data 998 are provided below). For example, associater 1120 may search data 998 for one or more functional domains associated with each alternative splice variant sequence and assign one or more putative functions, based at least in part upon ontological terms associated with these functional domains. In an illustrative, non-limiting example, associater 1120 associates at least one of the one or more functional domains associated with a particular alternative splice variant sequence with an ontological term “kinase” based, at least in part, upon the presence of the same or similar composition of one or more functional domains associated with the ontological term in data 998. Associater 1120 may thus provide one or more putative functions associated with the “kinase” ontological term.

As will now be appreciated by those of skill in the art, numerous other examples are possible and also numerous ontological classifications may be employed. It will also be appreciated that one or more ontological terms may be associated with each alternative splice variant sequence. Additionally, each of the alternative splice variant sequences may be analyzed by what is known to those of ordinary skill in the art as ‘clustering’, based upon these associated ontological terms.

Associater 1120 may provide each alternative splice variant sequence, one or more associated functional domains, and one or more putative functions to output manger 534 or functional domains analyzer 1130.

Analyzer 1130 may analyze data provided by associater 1120 for variation in functional domain composition and arrangement. In an illustrative, non-limiting and non-restrictive example, analyzer 1130 may identify variation in functional domain composition and arrangement associated with each alternative splice variant sequence with respect to at least one other alternative splice variant sequence. In the present example, the variation may include the presence or absence, relative position, and/or redundancy of at least one functional domain in at least one of a plurality of alternative splice variant sequences.

Additionally, analyzer 1130 may access one or more databases, such as database 518, to obtain additional information pertaining to the alternative splice variant sequences and associated functional domains. Analyzer 1130 may provide all information associated with each alternative splice variant sequence to output manger 534.

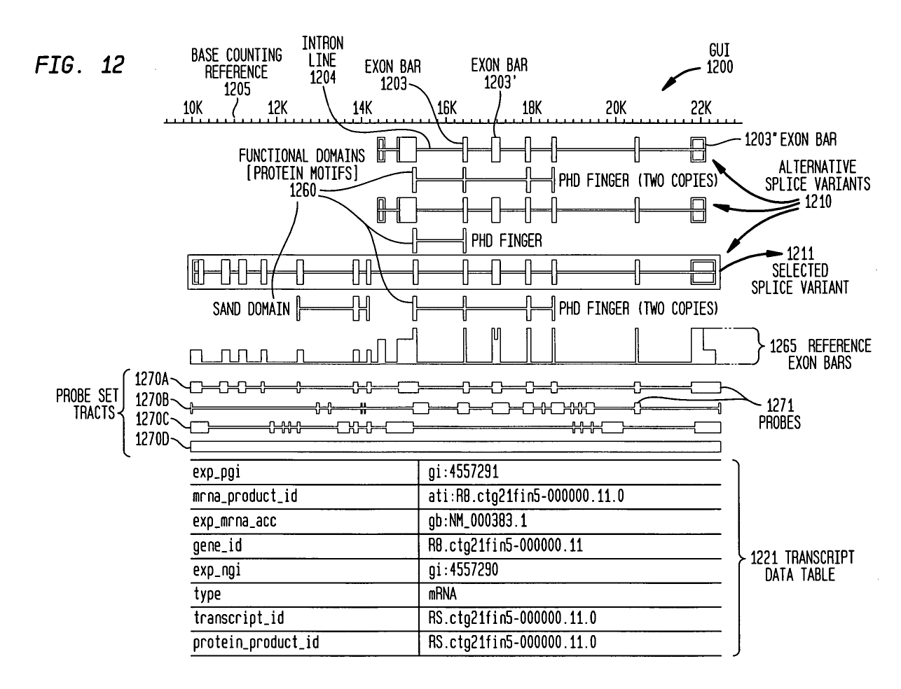

FIG. 12 is an illustrative example of a graphical user interface providing user 101 with information obtained by functional analysis of alternative splice variant sequences. It will be appreciated by those of ordinary skill in the relevant art that numerous alternative formats, both textual and graphical, may be used in other implementations. FIG. 12 shows GUI 1200, described below in detail, which displays exon bars 1203, 1203′, 1203″ and other related elements. Additionally, GUI 1200 may display elements such as protein functional domains 1260 associated with the alternative splice variant sequences. Information regarding the sequences, locations, homology, functions, two-dimensional or three-dimensional structure, and other aspects of protein functional domains or modules may, for example, be obtained in the manner described above from numerous remote databases 402 that, for instance, may include BLOCKS, InterPRO, eMotif, SCOP, HMM based database and search services including TM-HMM, Smart, Pfam, and NCBI CDD web-based databases and similar databases that may be developed in the future. Additional aspects of data collection and characterization regarding functional domains of proteins and protein-protein interactions are described in U.S. Provisional Patent Application No. 60/385,626, filed Jun. 4, 2002, titled “System, Method, and Product for Predicting Protein Interactions,” which is hereby incorporated herein by reference in its entirety for all purposes.

Functional domains 1260 displayed in GUI 1200 may vary according to the composition of alternative splice variant sequences. In this illustrative non-limiting example, one or more functional domains associated with the alternative splice variants 1210 are graphically aligned below the representation of the corresponding alternative splice variant. In the present example, each functional domain may be represented by one or more vertical bars or a combination of a plurality of such bars. It may noted that, in the present context, the terms “alternative splice variants” and “alternative splice variant sequences” are used broadly, in a non-limiting and non-restrictive manner and generally refer to biological sequences formed as result of alternative splicing as described above.

In some implementations, one or more elements of GUI 1200 may be interactive. For example, user 101 may click or select one or more domains 1260 to display additional related information in the same or different GUIs. Additional examples of visualizing alternative splice variants are provided in U.S. Provisional Patent Applications Nos. 60/394,574 and 60/375,875, incorporated by reference above.

In some implementations, GUI 1200 may display information relating to a common biological sequence that, for instance, may include a gene from which the alternative splice variants 1210 are derived. Such information could include gene name, protein name, accession numbers, protein ID numbers, splice variants ID's, numbers of variants, variant function, as well as other related genomic and/or experimental information. In some implementations, GUI 1200 may display such information in a tabular format, related specifically to a splice variant selected by the user. The tabular format may include one or more transcript data tables 1221. The information in table 1221 may be user interactive and include links to local and/or remote databases or resources such as, for example, by hyperlink to genomic information over the Internet. User 101 may select all or part of one or more splice variants by a variety of methods known to those of ordinary skill in the related art. In the illustrative example of FIG. 12 a user selection of an alternative splice variant sequence is displayed as selected splice variant 1211. In the present example, selected splice variant 1211 may include one or more elements of GUI 1200.

GUI 1200 displays alternative splice variants 1210 aligned to a scale illustrated in FIG. 12 as base counting reference 1205. Reference 1205 may include a variety of scales that may vary in units and magnitude including linear, logarithmic, and other types of scales. The alternative splice variant and/or gene aligned in this manner may have been selected by a user in accordance with any of the techniques noted herein. In some implementations, each alternative splice variant may be distinguished from the others by displaying each alternative splice variant along a separate horizontal line, i.e., by separating the variants vertically in GUI 1200. However, it will be understood that many other graphical arrangements or devices known to those of skill in the art may be used to distinguish splice variants and/or distinguish exons belonging to one or more splice variants. For example, the variants and/or their exons may be color-coded, identified by differently shaped objects, arranged differently and so on.

Base-counting reference 1205 may display a scale that may include a range of bases (or other residues in alternative implementations). Initial or other reference points determining the scale of reference 1205 may be user selectable so that, for example, bases may be counted from the beginning of a gene of interest chosen by user 101 (or a particular regulatory or other site related to the gene), the beginning or other reference point on a chromosome that includes the gene of interest, and so on.

As mentioned earlier, the exonic regions may be represented as vertical bars or boxed regions, for example, exon bars 1203, 1203′ and 1203″. The intronic regions may be represented by lines, for example, intron line 1204. For example, untranslated exons may be displayed as unfilled or empty boxes such as, for example, exon bar 1203″. Additionally, the translated exons, translated in different frames may be represented by differently colored bars. The foregoing examples are presented for the purposes of illustration only. Those of ordinary skill in the related art will appreciate that different representations may be used in other implementations such as, for instance, introns may be represented by vertical bars and exons may be represented by lines, additionally, different representations and/or coloring schemes may be used for representing exons.

In addition to providing an expanded view of a user-selected splice variant sequence or portion thereof, GUI 1200 in the illustrated example displays alternative splice variant sequences graphically aligned to one another and to one or more probe set tracts 1270A, 1270B, 1270C and 1270D. The probe set tracts 1270A to 1270D may represent parts or whole or combination of one or more different types of probe sets, for example, probe set tract 1270A may be comprised of one or more probe sets capable of detecting alternative splice variants, tract 1270B may be a part of probe set capable of preferentially detecting mRNA or other type of transcript, tract 1270C may be a part of user selected custom probe set, and tract 1270D may be a part of a probe set capable of detecting the ‘transcriptome’ or a substantial majority of transcripts present in a biological entity. The term “transcriptome” generally refers to the majority or all of the activated genes, mRNAs, or transcripts in a particular cell or tissue at a particular time.

Additionally, clicking or selecting of one or more variants 1210 or domains 1260 may alter one or more graphical characteristics of one or more probe set tracts 1270A to 1270D. In a non-limiting, illustrative example, clicking on or selecting one or more variants 1211 or domains 1260 may highlight or otherwise alter the display of one or more probes 1271 aligned with the user selection of variants 1211 or domains 1260. In the present example, one or more probes 1271 comprising the probe set tracts may identify all or part of the alternative splice variant sequences associated with aligned variants 1211 or domains 1260. In the present example, highlighted probes 1271 in the displayed probe sets may indicate the one or more probe sets, associated with the one or more probe set tracts, suitable for interrogating one or more regions of interest.

The foregoing are illustrative examples only and should not be construed as limiting or restrictive in any manner. Parts or tracts of many other types of probe sets, presently known or to become available in the future may be displayed, including one or more user selectable custom probe sets. Additionally, the information regarding any of the one or more probe set tracts and/or probes may be displayed in table 1221.

The probes comprising the one or more probe set tracts 1270 are shown illustratively as vertical bars 1271. In this non-limiting, illustrative example, length of the sequence of a probe may be shown as to be equal for all probes and may, for example, be 25 bases or ‘mers’ long. It may be noted that in some regions the probes are displayed as contiguous boxed regions and in this illustrative example, these contiguous regions do not represent length of the probes but may represent contiguous or overlapping probes or alternatively may represent probes that may not be contiguous but are significantly contiguous with minimal gaps. Furthermore, the sequences of one or more probes 1271 may represent sequences capable of binding to (or hybridizing with) alternative splice variants 1210. The probes may be capable of binding to exonic regions of alternative splice variants 1210.

The relative abundance of alternative splice variants may also be displayed in GUI 1200. Methods for representing abundance may include variations in exon bar height, variations in exon bar pattern, color coding of exon bars 1203, 1203′ and 1203″, or other graphical methods commonly used to distinguish differences. The measure of abundance could include the relative expression level of each alternative splice variant, the frequency of exon usage in all alternative splice variants, or other user-selected measure. For example, GUI 1200 includes reference exon bar 1265. The height of exon bar 1265 may correspond, as one of the examples noted above, to the frequency with which an exon, or partial exon, occurs in the alternative splice transcripts. In the present example, various bar heights may occur within each exon and between different exons.

The GUI 1200 in the illustrated implementation has what are referred by those in the related art as scroll bars. A user may interact with GUI 1200 by selecting a scroll bar and moving it in a desired direction to change what is displayed in the associated pane. For example, a user may select the vertical scroll bar associated with the GUI 1200 and move it in a desired direction. The one or more displayed alternative splice variant sequences displayed in GUI 1200 will change according to the direction of movement of the scroll bar as may the position of base counting reference 1205.

Additionally, a scroll bar or other method of selection could be used for what may be referred to as “semantic zooming”. This term as used herein refers to increasing or decreasing the levels of magnification and resolution in a display. With a change in magnification, objects may change appearance or shape as they change size. Moreover, when magnification of a displayed image is increased, additional information may be displayed relating to elements of the display. Conversely, when the magnification of an image is decreased, less information may be displayed for individual elements of the display. For example, when alternative splice variants are displayed at low magnification, the displayed image may include general exon structure and alignments. As the magnification is increased, the sequence of the alternative splice variants may be displayed as well as annotation information. Thus, not only is the magnification of the information changed, the amount, content, and/or type of information also may be changed in relation to the change of magnification. For a review of semantic and other zooming technology, see, e.g., CounterPoint: Creating Jazzy Interactive Presentations, Good, L., Bederson, B. B., HCIL-2001-3, CS-TR-4225, UMIACS-TR-2001-14, March 2001; Jazz: An Extensible Zoomable User Interface Graphics Toolkit in Java, Bederson, B., Meyer, J., Good, L. HCIL-2000-13, CS-TR-4137, UMIACS-TR-2000-30, May 2000, In ACM UIST 2000, pp. 171-180; Jazz: An Extensible 2D+Zooming Graphics Toolkit in Java Bederson, B., McAlister, B. HCIL-99-07, CS-TR-4015, UMIACS-TR-99-24, May 1999; Does Zooming Improve Image Browsing? Combs, T., T. A., and Bederson, B., HCIL-99-05, CS-TR-3995, UMIACS-TR-99-14, February 1999 In ACM Digital Library Conference, pp. 130-137; Graphical Multiscale Web Histories: A Study of PadPrints Hightower, R. R., Ring, L. T., Helfman, J. I., Bederson, B. B., and Hollan, J. D. ACM Conference on Hypertext 1999; Does Animation Help Users Build Mental Maps of Spatial Information, Bederson, B. and Boltman, A., CS-TR-3964, UMIACS-TR-98-73, September 1998, In IEEE Info Vis 99, pp. 28-35; A Zooming Web Browser, Bederson, B. B., Hollan, J. D., Stewart, J., Rogers, D., Vick, D., Ring, L. T., Grose, E., Forsythe, C. Human Factors in Web Development, Eds. Ratner, Grose, and Forsythe, Lawrence Erlbaum Assoc., pp 255-266, 1998; Implementing a Zooming User Interface: Experience Building Pad++, Bederson, B., Meyer, J., Software: Practice and Experience, 28 (10), pp. 1101-1135, August 1998; When Two Hands Are Better Than One:Enhancing Collaboration Using Single Display Groupware, Stewart, J., Raybourn, E. M., Bederson, B. B., Druin, A., ACM CHI 98 Summary, 1998; KidPad: A Design Collaboration Between Children, Technologists, and Educators, Druin, A., Stewart, J., Proft, D., Bederson, B. B., Hollan, J. D., ACM CHI 97, pp 463-470, 1997; A Multiscale Narrative: Gray Matters, Wardrip-Fruin, N., Meyer, J., Perlin, J., Bederson, B. B., Hollan, J. D., ACM SIGGRAPH 97 Visual Proceedings, p 141, 1997; A Zooming Web Browser, Bederson, B. B., Hollan, J. D., Stewart, J., Rogers, D., Drum, A., and Vick, D. SPIE Multimedia Computing and Networking, Volume 2667, pp 260-271, 1996; Local Tools: An Alternative to Tool Palettes, Bederson, B. B., Hollan, J. D., Druin, A., Stewart, J., Rogers, D., Proft, D., ACM UIST '96, pp 169-170, 1996; Pad++: A Zoomable Graphical Sketchpad for Exploring Alternate Interface Physics, Bederson, B., Hollan, J., Perlin, K., Meyer, J., Bacon, D., and Furnas, G., Journal of Visual Languages and Computing, 7, 3-31, 1996, HTML, Postscript without pictures (74K), PDF without pictures (77K) 1995; Space-Scale Diagrams: Understanding Multiscale Interfaces, Furnas, G., Bederson, B., ACM SIGCHI '95; Advances in the Pad++Zoomable Graphics Widget, Bederson, B., Hollan, J. USENIX Tcl/Tk'95 Workshop; Pad++: Advances in Multiscale Interfaces, Bederson, B. B., Stead, L., Hollan, J. D. ACM SIGCHI '94 (short paper), 1994; Pad++: A Zooming Graphical Interface for Exploring Alternate Interface Physics, Bederson, B. B., Hollan, J. D., ACM UIST '94, 1994; Pad—An Alternative Approach to the Computer Interface, Perlin, K., Fox, D., ACM SIGGRAPH '93; A Multiscale Approach to Interactive Display Organization, Perlin, K., Coordination Theory and Collaboration Technology Workshop, National Science Foundation, June 1991, each of which is hereby incorporated by reference herein in their entireties for all purposes.

Additional interactive features of GUI 1200 may include selecting elements such as an exon bar 1203, 1203′ or 1203″ by moving a cursor via mouse or keyboard and clicking the button on the mouse, or pressing the enter key on the keyboard, or other method commonly used for selecting elements. When a user selects an element or elements, portal 400 may alter the display in the graphical user interface and/or present one or more additional graphical user interfaces, or windows.

One of many possible examples of the utility of these features includes a situation in which user 101 inputs probe set identifiers or nucleotide sequences for which there are no known corresponding probe sets. Following this determiner 820 formulates a query to database manager 512 to determine alternative splice variants that are known to correspond to the input set of one or more probe-set identifiers provided by the user. Correlator 830 may formulate a query via database manager 512 to database 513 to obtain links to appropriate information located in local genomic database 518. The information used to establish this association may be predetermined based on expert input and/or computer-implemented analysis (e.g., statistical and/or by an adaptive system such as a neural network) of the nature of inquiries by users. This information may include data regarding translation of nucleotide sequences of the alternative splice variants to protein sequences, annotation data related to the splice variants, and other data regarding clustering of alternative splice variants. These and similar processes are represented by step 725 of FIG. 7.

Functional domains associater 1120, of alternative splice variant analyzer 840, may determine the functional domains associated with alternative splice variants as described above. It will be appreciated that that not all alternative splice variants have one or more functional domains associated with them. It is possible that one or more alternative splice variants may have no known functional domain associated with them, this may especially be true if the one or more alternative splice variants are newly discovered or were unknown earlier. Associater 1120 may putatively associate one or more functional domains with such alternative splice variants and this information may then be stored in one or more databases 518. These and similar processes are represented by step 735 of FIG. 7.

Functional domains analyzer 1130 may analyze the differences in functional domains associated with alternative splice variants as described above and forward the results of this analysis to output manger 534 for further processing, as represented by step 740 of FIG. 7. Output manager 534 may prepare and display the results received from analyzer 1130 in one or more GUIs 1200, as represented by step 745 of FIG. 7. It may be noted here that, as also mentioned above, the term “functional domain” is used broadly and generally refers to annotation data pertaining to the associated “functional domains” in the present context, wherein annotation data includes, but is not limited to, annotation terms, sequences and so on.

Furthermore, additional information provided by associater 1120 and/or analyzer 1130 to manager 534 may include ontological information associated with alternative splice variants and/or their associated functional domains, as represented by Ontological functional domain correlation and clustering data 998.

Data 998 is described herein with reference to a particular widely used scheme and program, developed and maintained by the Gene Ontology™ (GO) Consortium, for providing biological knowledge and genetic ontological information in particular. Biological knowledge, as used herein, refers to information that describes function (e.g., at molecular, cellular and system levels), structure, pathological roles, toxicological implications, and so on. It will be understood that although the GO system is illustratively referred to herein, various other systems for providing biological knowledge and genetic ontological information, such as the MGED Ontology system, may be employed in alternative implementations. At the core of the GO system is a dynamic controlled vocabulary for molecular biology that may be applied to all organisms and may be updated as biological information accumulates and changes. Further information about GO may be found in Gene Ontology: tool for the unification of biology, Nature Genet. 25: 25-29 (the Gene Ontology Consortium, 2000). Access to this ontological system, and information about it, are currently available over the Internet at http://www.geneontology.org/. Additional details and methods that may be employed for representing and displaying such data are described in U.S. patent application Ser. No. 10/328,872, titled “METHOD SYSTEM AND COMPUTER SOFTWARE FOR PROVIDING GENOMIC ONTOLOGICAL DATA”, filed Dec. 23, 2002, and hereby incorporated by reference in its entirety for all purposes.

Additional interactive features of GUI 1200 may include selecting at least one of a graphical elements by moving a cursor via mouse or keyboard and clicking the button on the mouse, or pressing the enter key on the keyboard, or other method commonly used for selecting elements. When a user selects an element or elements, portal 400 may alter the display in the graphical user interface and/or present one or more additional graphical user interfaces, or windows. Furthermore, user 101 may select or click on one or more probe set tracts 1270A to 1270D and obtain information including the arrays on which the selected probe sets are available and may then place an order for one or more arrays via portal 400. Additional details are described in U.S. patent application Ser. No. 10/328,818, titled “METHOD SYSTEM AND COMPUTER SOFTWARE FOR PROVIDING MICROARRAY PROBE DATA”, filed Dec. 23, 2002 and hereby incorporated by reference in its entirety for all purposes.

As will now be appreciated by those of ordinary skill in the relevant art in light of this disclosure, the above described graphical user interface may be used as a tool to display a very wide range of information, including biological information, that lends itself to linear comparison and visualization. Furthermore, the above mentioned description is illustrative only and does not limit the invention any way whatsoever. Additionally, in the above description the graphical elements of the graphical user interface described above are for illustrative purposes only and one or more graphical elements may be lacking in some implementations.

As used herein, the term “graphical user interface” is intended to be broadly interpreted so as to include various ways of communicating information to, and obtaining information from, a user. For example, information may be sent to a user in an email as an alternative to, or in addition to, presenting the information on a computer screen employing graphical elements (such as shown illustratively in FIG. 12). As is known by those of ordinary skill in the relevant art, the email may include graphics, or be designed to invoke graphics; similar to those that may be displayed in an interactive graphical user interface.

As indicated above, functional elements of portal 400 may be implemented in hardware, software, firmware, or any combination thereof. In the embodiment described above, it generally has been assumed for convenience that the functions of portal 400 are implemented in software. That is, the functional elements of the illustrated embodiment comprise sets of software instructions that cause the described functions to be performed. These software instructions may be programmed in any programming language, such as Java, Perl, C++, another high-level programming language, low-level languages, and any combination thereof. The functional elements of portal 400 may therefore be referred to as carrying out “a set of genomic web portal instructions,” and its functional elements may similarly be described as sets of genomic web portal instructions for execution by servers 510, 520, and 530.

In some embodiments, a computer program product is described comprising a computer usable medium having control logic (computer software program, including program code) stored therein. The control logic, when executed by a processor, causes the processor to perform functions of portal 400 as described herein. In other embodiments, some such functions are implemented primarily in hardware using, for example, a hardware state machine. Implementation of the hardware state machine so as to perform the functions described herein will be apparent to those skilled in the relevant arts.

Aspects of probe selection and design and other features applicable to implementations of the present invention are described in greater detail in U.S. patent application Ser. Nos. 10/028,884, 10/027,682, 10/028,416, and 10/006,174, all of which are hereby incorporated by reference herein in their entireties for all purposes.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways and by various elements in alternative embodiments. For example, some or all of the functions described as being carried out by determiner 820 could be carried out by correlator 830, or these functions could otherwise be distributed among other functional elements. Also, the functions of several elements may, in alternative embodiments, be carried out by fewer, or a single, element. For example, the functions of determiner 820 and correlator 830 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the division of functions between an application server and an internet server of the genome portal is illustrative only. The functions performed by the two servers could be performed by a single server or other computing platform, distributed over more than two computer platforms, or other otherwise distributed in accordance with various known computing techniques.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel or distributed processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A system for analysis of alternative splice variant sequences, comprising:
an input manager constructed and arranged to receive at least two alternative splice variant sequences, wherein the at least two alternative splice variant sequences are identified by one or more probe sets;
a correlator constructed and arranged to correlate one or more functional domains with each of the at least two alternative splice variant sequences; and
an associater constructed and to associate one or more putative functions with each of the at least two alternative splice variant sequences based, at least in part, upon a combination of the one or more functional domains.

2. The system of claim 1, wherein:
the one or more probe sets are identified by one or more probe set identifiers.

3. The system of claim 1, wherein:
each of the one or more functional domains includes one or more sequences that share one or more measures of similarity between each of the at least two alternative splice variant sequences.

4. The system of claim 1, wherein:
the one or more functional domains are identified by one or more probe sets associated with each of the at least two alternative splice variant sequences.

5. The system of claim 1, wherein:
the one or more functional domains include one or more protein motifs.

6. The system of claim 5, wherein:
the one or more protein motifs include a zinc finger, a PHD finger, a SAND domain, a G protein-coupled receptor, a FYVE finger, a kinesin and a SANT domain.

7. The system of claim 1, wherein:
the putative functions include one or more functions associated with one or more ontological terms.

8. A system, comprising:
an input manager constructed and arranged to receive a plurality of probe set identifiers and associated intensity values;
a determiner constructed and arranged to determine at least two alternative splice variant sequences based, at least in part, upon the one or more probe set identifiers and associated intensity values;
a correlator constructed and arranged to correlate one or more functional domains with each of the at least two alternative splice variant sequences;
an associater constructed and arranged to associate one or more putative functions with each of the at least two alternative splice variant sequences based, at least in part, upon a combination of the one or more functional domains; and
an output manager constructed and arranged to display the putative functions in one or more graphical user interfaces.

9. The system of claim 8, wherein:
the one or more probe sets are identified by one or more probe set identifiers.

10. The system of claim 8, wherein:
each of the one or more functional domains includes one or more sequences that share one or more measures of similarity between each of the at least two alternative splice variant sequences.

11. The system of claim 8, wherein:
the one or more functional domains are identified by one or more probe sets associated with each of the at least two alternative splice variant sequences.

12. The system of claim 8, wherein:
the one or more functional domains include one or more protein motifs.

13. The system of claim 12, wherein:
the one or more protein motifs include a zinc finger, a PHD finger, a SAND domain, a G protein-coupled receptor, a FYVE finger, a kinesin and a SANT domain.

14. The system of claim 8, wherein:
the putative functions include one or more functions associated with one or more ontological terms.

15. A system, comprising:
an input manager constructed and arranged to receive at least two alternative splice variant sequences;
a correlator constructed and arranged to correlate one or more functional domains with each of the at least two alternative splice variant sequences;
a analyzer constructed and arranged to compare one or more differences between each of the at least two alternative splice variant sequences based, at least in part, upon the one or more functional domains; and
an output manager constructed and arranged to display the one or more differences of each of the at least two alternative splice variant sequences in one or more graphical user interfaces.

16. The system of claim 15, wherein:
the one or more probe sets are identified by one or more probe set identifiers.

17. The system of claim 15, wherein:
each of the one or more functional domains includes one or more sequences that share one or more measures of similarity between each of the at least two alternative splice variant sequences.

18. The system of claim 15, wherein:
the one or more functional domains are identified by one or more probe sets associated with each of the at least two alternative splice variant sequences.

19. The system of claim 15, wherein:
the one or more functional domains include one or more protein motifs.

20. The system of claim 19, wherein:
the one or more protein motifs include a zinc finger, a PHD finger, a SAND domain, a G protein-coupled receptor, a FYVE finger, a kinesin and a SANT domain.

21. A system, comprising:
an application server comprising an input manager constructed and arranged to receive at least two alternative splice variant sequences, wherein the at least two alternative splice variant sequences are identified by one or more probe sets; a correlator constructed and arranged to correlate one or more functional domains with each of the at least two alternative splice variant sequences; and an associater constructed and to associate one or more putative functions with each of the at least two alternative splice variant sequences based, at least in part, upon a combination of the one or more functional domains; and
an internet server comprising an output manager constructed and arranged to display the putative functions in one or more graphical user interfaces.

22. The system of claim 21, wherein:
the internet server further comprises an input manger constructed and arranged to receive user input; and
the system further comprises one or more user computers constructed and arranged to enable a user to provide a user selection of one or more alternative splice variant sequences to the internet server.

23. The system of claim 21, wherein:
the output manager provides the graphical user interfaces via the internet.

24. The system of claim 21, wherein:
the one or more probe sets are identified by one or more probe set identifiers.

25. The system of claim 21, wherein:
each of the one or more functional domains includes one or more sequences that share one or more measures of similarity between each of the at least two alternative splice variant sequences.

26. The system of claim 21, wherein:
the one or more functional domains are identified by one or more probe sets associated with each of the at least two alternative splice variant sequences.

27. The system of claim 21, wherein:
the one or more functional domains include one or more protein motifs.

28. The system of claim 27, wherein:
the one or more protein motifs include a zinc finger, a PHD finger, a SAND domain, a G protein-coupled receptor, a FYVE finger, a kinesin and a SANT domain.

29. The system of claim 21, wherein:
the putative functions include one or more functions associated with one or more ontological terms.

30. A system, comprising:
means for receiving at least two alternative splice variant sequences, wherein the at least two alternative splice variant sequences are identified by one or more probe sets;
means for correlating one or more functional domains with each of the at least two alternative splice variant sequences; and
means for associating one or more putative functions with each of the at least two alternative splice variant sequences based, at least in part, upon a combination of the one or more functional domains.

31. A method for analysis of alternative splice variant sequences, comprising the acts of:
receiving at least two alternative splice variant sequences, wherein the at least two alternative splice variant sequences are identified by one or more probe sets;
correlating one or more functional domains with each of the at least two alternative splice variant sequences; and
associating one or more putative functions with each of the at least two alternative splice variant sequences based, at least in part, upon a combination of the one or more functional domains.

32. The method of claim 31, wherein:
the one or more probe sets are identified by one or more probe set identifiers.

33. The method of claim 31, wherein:
each of the one or more functional domains includes one or more sequences that share one or more measures of similarity between each of the at least two alternative splice variant sequences.

34. The method of claim 31, wherein:
the one or more functional domains are identified by one or more probe sets associated with each of the at least two alternative splice variant sequences.

35. The method of claim 31, wherein:
the one or more functional domains include one or more protein motifs.

36. The method of claim 35, wherein:
the one or more protein motifs include a zinc finger, a PHD finger, a SAND domain, a G protein-coupled receptor, a FYVE finger, a kinesin and a SANT domain.

37. The method of claim 31, wherein:
the putative functions include one or more functions associated with one or more ontological terms.

38. A method comprising the acts of:
receiving a plurality of probe set identifiers and associated intensity values;
determining at least two alternative splice variant sequences based, at least in part, upon the one or more probe set identifiers and associated intensity values;
correlating one or more functional domains with each of the at least two alternative splice variant sequences;
associating one or more putative functions with each of the at least two alternative splice variant sequences based, at least in part, upon a combination of the one or more functional domains; and
displaying the putative functions in one or more graphical user interfaces.

39. The method of claim 38, wherein:
the one or more probe sets are identified by one or more probe set identifiers.

40. The method of claim 38, wherein:
each of the one or more functional domains includes one or more sequences that share one or more measures of similarity between each of the at least two alternative splice variant sequences.

41. The method of claim 38, wherein:
the one or more functional domains are identified by one or more probe sets associated with each of the at least two alternative splice variant sequences.

42. The method of claim 38, wherein:
the one or more functional domains include one or more protein motifs.

43. The method of claim 42, wherein:
the one or more protein motifs include a zinc finger, a PHD finger, a SAND domain, a G protein-coupled receptor, a FYVE finger, a kinesin and a SANT domain.

44. The method of claim 38, wherein:
the putative functions include one or more functions associated with one or more ontological terms.

45. A method comprising the acts of:
receiving at least two alternative splice variant sequences;
correlating one or more functional domains with each of the at least two alternative splice variant sequences;
comparing one or more differences between each of the at least two alternative splice variant sequences based, at least in part, upon the one or more functional domains; and
displaying the one or more differences of each of the at least two alternative splice variant sequences in one or more graphical user interfaces.

46. The method of claim 45, wherein:
the one or more probe sets are identified by one or more probe set identifiers.

47. The method of claim 45, wherein:
each of the one or more functional domains includes one or more sequences that share one or more measure of similarity between each of the at least two alternative splice variant sequences.

48. The method of claim 45, wherein:
the one or more functional domains are identified by one or more probe sets associated with each of the at least two alternative splice variant sequences.

49. The method of claim 45, wherein:
the one or more functional domains include one or more protein motifs.

50. The method of claim 49, wherein:
the one or more protein motifs include a zinc finger, a PHD finger, a SAND domain, a G protein-coupled receptor, a FYVE finger, a kinesin and a SANT domain.
